(19) United States
(12) Patent Application Publication
(10) Pub. No.: US 2010/0286073 A1
(43) Pub. Date: Nov. 11, 2010

(54) COMPOUNDS AND METHODS FOR PHARMACEUTICAL USE

(76) Inventor: Paul JENKINS, Potters Bar Hertfordshire (GB)

Correspondence Address: WILMERHALE/BOSTON, 60 STATE STREET, BOSTON, MA, 02109, US

(21) Appl. No.: 12/681,425

(22) PCT Filed: Oct. 3, 2008
(86) PCT No.: PCT/GB2008/050893

§ 371 Date: Jul. 19, 2010

(30) Foreign Application Priority Data
Oct. 3, 2007 (GB) 0719248.7

(51) Int. Cl. A61K 31/495; C07D 295/096; A61P 25/24; A61P 29/00; A61P 25/00; C07C 229/32; A61K 31/198; A61K 31/221; C07C 327/36; C07C 233/05; A61K 31/16; C07D 207/448; A61K 31/4015; C07C 323/12; A61K 31/10; C07D 471/04; A61K 31/4745; C07C 65/10; A61K 31/60; C07D 209/20; A61K 31/405; C07C 217/54; A61K 31/138; C07F 9/10; A61K 31/6615; C07J 71/00; A61K 31/7048; C07D 339/04; A61K 31/385; C07F 9/09

(52) U.S. Cl. 514/26; 544/398; 514/252.12; 562/506; 514/561; 514/513; 558/254; 564/209; 514/629; 548/548; 514/425; 568/62; 514/706; 546/88; 514/292; 562/477; 514/159; 548/496; 514/419; 564/349; 514/652; 554/79; 514/103; 536/6; 549/39; 514/440; 544/243; 514/86

(57) ABSTRACT

The present invention relates to compounds, as well as to compositions, methods and kits comprising such compounds, for use in the treatment or prophylaxis of fibromyalgia, chronic fatigue syndrome, myofascial pain syndrome, Gulf War syndrome and related conditions, wherein the compound is a fatty acid oxidation inhibitor and/or a carbohydrate oxidation activator.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference in their entireties.

### FIELD OF THE INVENTION

The present invention relates generally to compositions and methods for the treatment of certain rheumatic conditions such as fibromyalgia syndrome (FMS), chronic fatigue syndrome (CFS), myofascial pain syndrome (MPS), and Gulf War syndrome (GWS), among others.

### BACKGROUND OF THE INVENTION

Fibromyalgia is a syndrome characterized by chronic and intense generalized pain over portions of the body. The pain is not limited to muscle tissue and may also be experienced in the skin. FMS is estimated to affect 2-5% of the population, and associated symptoms often include fatigue, malaise, depression, anxiety, muscle tightness in the morning, muscle stiffness, and sleep disorders. FMS is characterized by a generalized heightened perception of sensory stimuli. Other symptoms may include headaches, facial pain, cognitive impairment, gastrointestinal complaints, frequent urination, diarrhea, constipation and dysmenorrhea.

Various theories exist as to possible causes of FMS, although no agreement exists within the medical community, and at present, the causes and mechanisms triggering the onset of FMS are not known. Fibromyalgia has been recognized only since the 1980s as a medical disorder, and its proper diagnosis is complicated by its diverse symptoms and the lack of definitive diagnostic tests, e.g., immunological, virological, pathological, and the like, for identifying the condition. In general, FMS is one of a number of poorly understood illnesses, including chronic fatigue syndrome (CFS), myofascial pain syndrome (MPS), and others. A number of medications have been shown to have some degree of effectiveness in randomized clinical trials of patients with fibromyalgia, including antidepressants such as amitriptyline, duloxetine, fluoxetine, paroxetine, and milnacipran; muscle relaxants such as cyclobenzaprine; and certain analgesics such as tramadol. Currently, only pregabalin (Lyrica®) has been approved by the FDA for treating fibromyalgia, and FMS patients often suffer for years from widespread pain.

CFS is a disorder characterized by fatigue of an incapacitating nature lasting at least six months. CFS can affect virtually every major system in the body, including neurological, immunological, hormonal, gastrointestinal, and musculoskeletal (Friedberg F, et al., “Understanding Chronic Fatigue Syndrome: An Empirical Guide to Assessment and Treatment”, Washington D.C., American Psychological Association, 1998; Fukuda K, et al., Ann Intern Med, 1994, 121: 953-9). In addition to experiencing severe chronic fatigue, patients often exhibit several of the following symptoms: substantial impairment in short term memory, sore throat, tender lymph nodes, muscle pain, multi-joint pain without swelling or redness, headaches, unrefreshing sleep, and post-exertional malaise. Similar to FMS, definitive diagnostic markers or laboratory tests are unavailable to distinguish CFS. Thus, clinical diagnosis is largely based on self-reported symptoms and behavioral criteria. Relatively few patients with CFS are reportedly cured; a review by Joyce et al. reports that fewer than 10% of patients return to pre-illness levels of functioning following treatment (Joyce J, et al., Q J Med, 1997, 90: 223-33).

Many of the symptoms associated with CFS are also characteristic of other similar conditions, such as FMS and MPS. Several studies have suggested that CFS and FMS have many similarities (Buchwald D, Rheum Dis Clin North Am, 1996, 22: 219-43; Goldenberg D L, J Rheumatol, 1988, 15: 992-6). In fact, it has been estimated that 20% to 70% of patients with FMS meet the criteria for CFS and that about 35% to 75% of patients with CFS also have FMS (Buchwald D, et al., Arch Intern Med, 1994, 154: 2049-53; Hudson J I, et al., Am J Med, 1992, 92: 363-7). Typical treatment approaches for CFS include the administering of low doses of drugs directed to treatment of the symptoms experienced by the individual patient. Such drugs include tricyclic agents such as doxepin, amitriptyline, and nortriptyline, for improving sleep and relieving mild generalized pain; antidepressants such as fluoxetine, sertraline, and paroxetine, among others; anxiolytic agents to treat panic disorder such as alprazolam, clonazepam, and lorazepam; and non-steroidal anti-inflammatory drugs (NSAIDs) for relieving pain and fever such as naproxen, ibuprofen, and piroxicam. Additionally, antihypotensive agents such as fludrocortisone and beta blockers such as atenolol have been prescribed. Again, although many different approaches have been used to treat CFS, no single therapeutic agent or combination of therapeutic agents has been found to be significantly effective in the treatment of CFS.

Chronic myofascial pain (CMP), also referred to as myofascial pain syndrome (MPS), is a condition involving localized myofascial pain caused by trigger points. Myofascial patients commonly radiate pain from trigger points which are considered to be active. A myofascial trigger point is a localized area that is starving for oxygen. This results in release of neuroreactive biochemicals which sensitize nearby nerves. The sensitized nerves then initiate the motor, sensory, and autonomic effects of myofascial trigger points by acting on the central nervous system. Myofascial trigger points can be identified and documented electrophysiologically. They may also be identified histologically by contraction knots. Trigger points in MPS patients may be latent (non-symptomatic) or active (producing pain, at rest or with motion or loading to the muscle). Latent trigger points are typically activated by intense heat or cold, changing or damp weather, repetitive injury, and weekend athletic syndrome. Additional factors leading to vulnerability towards MPS include short leg syndrome, small hemipelvis, poor posture, prolonged immobility, vitamin and mineral deficiencies, endocrine dysfunctions, intense emotional stress, and poor work habits (Travell J F and Simmons D G, Myofascial Pain & Dysfunction: The Trigger Point Manual, VI & 2, Baltimore, Williams & Wilkins). Myofascial pain syndrome is often misdiagnosed as fibromyalgia. Current treatment approaches include physical therapy, trigger point injection with a local anesthetic, and the administration of drugs such as NSAIDs (e.g., ibuprofen), tricyclic antidepressants (e.g., amitriptyline), muscle relaxants (e.g., cyclobenzaprine), non-narcotic analgesics (e.g., tramadol), and anticonvulsants (e.g., gabapentin). Similar to the above conditions, while certain treatments can provide a degree of relief for certain patients, there currently exists no widely-accepted therapeutic approach for the effective treatment of MPS.

Often, many of the above conditions are misdiagnosed, or even undiagnosed. Currently existing therapies for the treatment of FMS, CFS, and MPS, have been of a limited, if any, well-accepted degree of success. Patients suffering from one or more of the foregoing conditions typically endure significant disabilities in terms of physical, occupational, and social functioning. Based upon the on-going research focused on these conditions, it can be seen that there is a need for an effective therapy for the treatment of FMS, CFS, MPS, and similar disorders. In particular, there is a need for compositions and treatments effective to significantly ameliorate or ideally, eliminate, one or more symptoms associated with one or more of the above conditions. It is believed that the present invention meets this need.

### SUMMARY OF THE INVENTION

Accordingly, in one aspect, the invention provides a method for the treatment of conditions such as fibromyalgia syndrome, chronic fatigue syndrome, and myofascial pain syndrome, among others.

The inventors arrived at the present discovery (forming the basis of the invention) in a completely unexpected fashion. While treating a subject suffering from a cardiac condition with an anti-anginal agent, trimetazidine, the inventors discovered that, within a short time after commencement of treatment, that same subject, also suffering from fibromyalgia syndrome, experienced a surprising and remarkable improvement in all symptoms associated with his fibromyalgia. This effect was later further confirmed in additional clinical studies of fibromyalgia patients for whom currently existing treatments for fibromyalgia had previously been either minimally or completely ineffective.

Trimetazidine (1-(2,3,4-trimethyloxybenzyl)piperazine) is a metabolic anti-anginal and anti-ischaemic agent that is used in the treatment of patients with coronary heart disease and stable angina. Unlike the conventional anti-anginal agents, which act by producing haemodynamic changes to restore balance between myocardial oxygen supply and demand, trimetazidine increases cellular tolerance to ischaemia.

Trimetazidine is thought to act by inhibiting mitochondrial fatty acid metabolism and secondarily by stimulating glucose metabolism (Kantor, et al., Circ Res, 2000, 86: 580-8). In particular, it has been shown that trimetazidine inhibits the long-chain isoform of an enzyme involved in mitochondrial fatty acid beta-oxidation, 3-ketoacyl coenzyme A thiolase (3KCT), thereby reducing fatty acid oxidation. An associated increase in pyruvate dehydrogenase activity stimulates glucose oxidation. This is thought to be due to the relief of fatty acid-induced inhibition of pyruvate dehydrogenase.

Thus, based on the surprising discovery that trimetazidine can be used to treat fibromyalgia and related conditions, the inventors have deduced that any substance that acts in the same manner, i.e. that inhibits fatty acid oxidation and/or stimulates carbohydrate metabolism, should also be effective in treating fibromyalgia and related conditions.

Accordingly, a first aspect of the present invention provides a compound, or a pharmaceutically acceptable salt, prodrug or hydrate thereof, for use in the treatment or prophylaxis of
(i) fibromyalgia;
(ii) chronic fatigue syndrome;
(iii) myofascial pain syndrome;
(iv) Gulf War syndrome;
(v) multiple chemical sensitivity;
(vi) widespread pain of at least three anatomical sites of a mammalian subject's body;
(vii) the symptom of unexplained fatigue which is persisting or relapsing;
(viii) the symptom of pain in one or more trigger points; and/or
(ix) one or more symptoms, in a subject that is a veteran of the Gulf War, selected from the group consisting of aching muscles, spasm, fatigue, irritability, thick saliva, weight loss, diarrhoea, skin rash, memory loss, dizziness, peripheral numbness, sleep disturbance, chronic fever, laboured breathing, and headache;
   wherein the compound, pharmaceutically acceptable salt, prodrug or hydrate is a fatty acid oxidation inhibitor and/or a carbohydrate oxidation activator.

In a preferred embodiment of the first aspect of the present invention, the fatty acid oxidation inhibitor is a mitochondrial fatty acid oxidation inhibitor, more preferably a beta-oxidation inhibitor, such as an inhibitor of acyl-CoA dehydrogenase, enoyl-CoA hydratase, 3-hydroxyacyl-CoA dehydrogenase or 3-ketoacyl-CoA thiolase.

Inhibitors of acyl-CoA dehydrogenase include hypoglycin, 3-chloro-3-butenoylpantetheine, 3-pentenoylpantetheine, iodoacetamide, N-ethylmaleimide, dithioerythritol, EDTA, o-phenanthroline, 2-mercaptoacetate, iodoacetic acid, 1-ethyl-3-(3-dimethylaminopropyl)-carbodiimide, diethyldicarbonate and pharmaceutically acceptable salts, prodrugs or hydrates thereof. A preferred inhibitor of acyl-CoA dehydrogenase is hypoglycin or a pharmaceutically acceptable salt, prodrug or hydrate thereof.

Inhibitors of enoyl-CoA hydratase include diethyldicarbonate, iodoacetamide, iodoacetic acid, N-ethylmaleimide, iodoacetate and pharmaceutically acceptable salts, prodrugs or hydrates thereof.

Inhibitors of 3-hydroxyacyl-CoA dehydrogenase include iodoacetamide, iodoacetic acid, N-ethylmaleimide, salicylic acid, 5,5′-dithiobis(2-nitrobenzoic acid), N-bromosuccinimide and pharmaceutically acceptable salts, prodrugs or hydrates thereof. A preferred inhibitor of 3-hydroxyacyl-CoA dehydrogenase is salicylic acid or a pharmaceutically acceptable salt, prodrug or hydrate thereof.

Most preferably the beta-oxidation inhibitor is an inhibitor of 3-ketoacyl-CoA thiolase. Inhibitors of 3-ketoacyl-CoA thiolase include iodoacetamide, N-ethylmaleimide, 4-pentenoic acid, 2-bromooctanoic acid, 4-bromocrotonic acid, 4-bromotiglic acid, trimetazidine, ranolazine, 4-bromo-2-octenoic acid, N-methylmaleimide, semicarbazide, tris(hydroxymethyl)aminomethane, benzotript and pharmaceutically acceptable salts, prodrugs or hydrates thereof. Preferred inhibitors of 3-ketoacyl-CoA thiolase include 4-pentenoic acid, 2-bromooctanoic acid, 4-bromocrotonic acid, 4-bromotiglic acid, trimetazidine, ranolazine and pharmaceutically acceptable salts, prodrugs or hydrates thereof. Most preferred inhibitors of 3-ketoacyl-CoA thiolase are trimetazidine, ranolazine and pharmaceutically acceptable salts, prodrugs or hydrates thereof.

In an alternative embodiment of the first aspect of the present invention, the fatty acid oxidation inhibitor is a mitochondrial fatty acid transport inhibitor, such as an inhibitor of CPT-1, CPT-2, carnitine-acylcarnitine translocase or γ-butyrobetaine hydroxylase.

Inhibitors of CPT-1 (carnitine palmitoyltransferase I) include etomoxir, oxfenicine, 4-hydroxyphenylglyoxylate, 2-tetradecylglycidic acid, trimetazidine derivative S-15176 (2,6-di-tert-butyl-4-{3-[4-(2,3,4-trimethoxybenzyl)-piperazin-1-yl]propylsulfanyl}phenol), metoprolol, perhexiline, aminocarnitine, amiodarone, diethyldicarbonate, 11-trimethylamino-undecanoyl-DL-carnitine, cardiolipin, carnitine, chenodeoxycholic acid, cholic acid, deoxycarnitine, digitonin, ethyl 2-[6-(4-nitrophenoxy)hexyl]oxirane-2-carboxylate, γ-linolenic acid, hemipalmitoylcarnitinium bromide, L-palmitoylcarnitine, L-sulfocarnitine, octyl glucoside, palmitoylcholine, phosphatidylcholine, 2-(2-naphthalen-2-yloxyethoxy)thiophene-5-glyoxylic acid, thiolcarnitine and pharmaceutically acceptable salts, prodrugs or hydrates thereof. Preferred inhibitors of CPT-1 include etomoxir, oxfenicine, 4-hydroxyphenylglyoxylate, 2-tetradecylglycidic acid, trimetazidine derivative S-15176, metoprolol, perhexiline, amiodarone and pharmaceutically acceptable salts, prodrugs or hydrates thereof.

Inhibitors of CPT-2 (carnitine palmitoyltransferase II) include perhexiline, aminocarnitine, diethyldicarbonate, 11-trimethylamino-undecanoyl-DL-carnitine, cardiolipin, carnitine, chenodeoxycholic acid, cholic acid, deoxycarnitine, digitonin, ethyl 2-[6-(4-nitrophenoxy)hexyl]oxirane-2-carboxylate, γ-linolenic acid, hemipalmitoylcarnitinium bromide, L-palmitoylcarnitine, L-sulfocarnitine, octyl glucoside, palmitoylcholine, phosphatidylcholine, 2-(2-naphthalen-2-yloxyethoxy)thiophene-5-glyoxylic acid, thiolcarnitine and pharmaceutically acceptable salts, prodrugs or hydrates thereof. Preferred inhibitors of CPT-2 include perhexiline, aminocarnitine and pharmaceutically acceptable salts, prodrugs or hydrates thereof.

Inhibitors of carnitine-acylcarnitine translocase include 2-(3-methyl-cinnamyl-hydrazono)propionate, 2-(3-phenylpropoxyimino)butyric acid, medium and long-chain acyl carnitines, BM 13.907 and pharmaceutically acceptable salts, prodrugs or hydrates thereof.

Inhibitors of γ-butyrobetaine hydroxylase include carnitine, iodoacetate, N-ethylmaleimide, 3-(2,2,2-trimethylhydrazine)propionate, 2-oxoglutarate, 3,4-dihydroxybenzoate, 3-(2,2-dimethylcyclopropyl)propionic acid, 3-bromo-2-oxoglutarate, 3-glutathione-2-oxoglutarate, 3-trimethylaminopropyl-1-sulfonate, 2,2′-bipyridine, ascorbate, dioxane, riboflavin-5′-phosphate, iodosobenzoate, pyridine-2,4-dicarboxylate, quinacrine, succinic semialdehyde and pharmaceutically acceptable salts, prodrugs or hydrates thereof. A preferred inhibitor of γ-butyrobetaine hydroxylase is 3-(2,2,2-trimethylhydrazine)propionate or a pharmaceutically acceptable salt, prodrug or hydrate thereof.

In a further embodiment of the first aspect of the present invention, the carbohydrate oxidation activator is a glucose oxidation activator, such as a glycolysis activator, a pyruvate dehydrogenase activator or a pyruvate dehydrogenase kinase inhibitor.

Compounds which activate pyruvate dehydrogenase and/or inhibit pyruvate dehydrogenase kinase include N-ethylmaleimide, dichloroacetate, 3,3,3-trifluoro-2-hydroxy-2-methylpropionamide, 2-chloroisohexanoate, 2-oxobutyrate, dichloroacetophenone, dihydrolipoic acid, 5,5′-dithiobis(2-nitrobenzoic acid), 3,3-dichloro-2-benzofuran-1-one, pyruvamide, lipoic acid, rapamycin, thiamine diphosphate, dobutamine and pharmaceutically acceptable salts, prodrugs or hydrates thereof. A preferred compound of this type is dichloroacetate or a pharmaceutically acceptable salt, prodrug or hydrate thereof.

In any embodiment of the first aspect of the present invention, the compound, pharmaceutically acceptable salt, prodrug or hydrate may be selected from hypoglycin, 3-chloro-3-butenoylpantetheine, 3-pentenoylpantetheine, iodoacetamide, N-ethylmaleimide, dithioerythritol, EDTA, o-phenanthroline, 2-mercaptoacetate, iodoacetic acid, 1-ethyl-3-(3-dimethylaminopropyl)-carbodiimide, diethyldicarbonate, iodoacetate, salicylic acid, 5,5′-dithiobis(2-nitrobenzoic acid), N-bromosuccinimide, 4-pentenoic acid, 2-bromooctanoic acid, 4-bromocrotonic acid, 4-bromotiglic acid, trimetazidine, ranolazine, 4-bromo-2-octenoic acid, N-methylmaleimide, semicarbazide, tris(hydroxymethyl)aminomethane, benzotript, etomoxir, oxfenicine, 4-hydroxyphenylglyoxylate, 2-tetradecylglycidic acid, trimetazidine derivative S-15176, metoprolol, perhexiline, amiodarone, aminocarnitine, 11-trimethylamino-undecanoyl-DL-carnitine, cardiolipin, carnitine, chenodeoxycholic acid, cholic acid, deoxycarnitine, digitonin, ethyl 2-[2-[6-(4-nitrophenoxy)hexyl]oxirane-2-carboxylate, γ-linolenic acid, hemipalmitoylcarnitinium bromide, L-palmitoylcarnitine, L-sulfocarnitine, octyl glucoside, palmitoylcholine, phosphatidylcholine, 2-(2-naphthalen-2-yloxyethoxy)thiophene-5-glyoxylic acid, thiolcarnitine, 2-(3-methyl-cinnamyl-hydrazono)propionate, 2-(3-phenylpropoxyimino)butyric acid, medium and long-chain acyl carnitines, BM 13.907, 3-(2,2,2-trimethylhydrazine)propionate, 2-oxoglutarate, 3,4-dihydroxybenzoate, 3-(2,2-dimethylcyclopropyl)propionic acid, 3-bromo-2-oxoglutarate, 3-glutathione-2-oxoglutarate, 3-trimethylaminopropyl-1-sulfonate, 2,2′-bipyridine, ascorbate, dioxane, riboflavin-5′-phosphate, iodosobenzoate, pyridine-2,4-dicarboxylate, quinacrine, succinic semialdehyde, dichloroacetate, 3,3,3-trifluoro-2-hydroxy-2-methylpropionamide, 2-chloroisohexanoate, 2-oxobutyrate, dichloroacetophenone, dihydrolipoic acid, 3,3-dichloro-2-benzofuran-1-one, pyruvamide, lipoic acid, rapamycin, thiamine diphosphate, dobutamine, or a pharmaceutically acceptable salt, prodrug or hydrate thereof. Preferably the compound, pharmaceutically acceptable salt, prodrug or hydrate is selected from hypoglycin, salicylic acid, 4-pentenoic acid, 2-bromooctanoic acid, 4-bromocrotonic acid, 4-bromotiglic acid, trimetazidine, ranolazine, etomoxir, oxfenicine, 4-hydroxyphenylglyoxylate, 2-tetradecylglycidic acid, trimetazidine derivative S-15176, metoprolol, perhexiline, amiodarone, aminocarnitine, 2-(3-methyl-cinnamyl-hydrazono)propionate, 2-(3-phenylpropoxyimino)butyric acid, medium and long-chain acyl carnitines, BM 13.907, 3-(2,2,2-trimethylhydrazine)propionate, dichloroacetate, or a pharmaceutically acceptable salt, prodrug or hydrate thereof. More preferably the compound, pharmaceutically acceptable salt, prodrug or hydrate is selected from 4-pentenoic acid, 2-bromooctanoic acid, 4-bromocrotonic acid, 4-bromotiglic acid, trimetazidine, ranolazine, or a pharmaceutically acceptable salt, prodrug or hydrate thereof. More preferably the compound, pharmaceutically acceptable salt, prodrug or hydrate is selected from trimetazidine, ranolazine, or a pharmaceutically acceptable salt, prodrug or hydrate thereof. In one embodiment of the first aspect of the present invention the compound, pharmaceutically acceptable salt, prodrug or hydrate is trimetazidine, in another embodiment the compound, pharmaceutically acceptable salt, prodrug or hydrate is a fatty acid oxidation inhibitor and/or a carbohydrate oxidation activator other than trimetazidine.

Inhibitors of acyl-CoA dehydrogenase, enoyl-CoA hydratase, 3-hydroxyacyl-CoA dehydrogenase, 3-ketoacyl-CoA thiolase, CPT-1, CPT-2, carnitine-acylcarnitine translocase, γ-butyrobetaine hydroxylase, and pyruvate dehydrogenase kinase, and activators of pyruvate dehydrogenase are known in the art, for example, from the BRENDA database (BRaunschweig ENzyme DAtabase), which is hereby incorporated herein by reference in its entirety.

A compound, pharmaceutically acceptable salt, prodrug or hydrate of the first aspect of the present invention may be for use in the treatment of
(i) fibromyalgia;
(ii) chronic fatigue syndrome;
(iii) myofascial pain syndrome;
(iv) Gulf War syndrome; and/or
(v) multiple chemical sensitivity;
   wherein the treatment results in a diminution of one or more major symptoms associated with said condition. Preferably the treatment results in at least a 50% improvement in said one or more major symptoms. Even more preferably the treatment results in at least a 90% improvement in said one or more major symptoms.

Where the compound, pharmaceutically acceptable salt, prodrug or hydrate is for use in the treatment of fibromyalgia, one of said major symptoms may be widespread pain of at least three anatomical sites of the subject's body. In such a case it is preferred that the treatment results in at least a 50% reduction in said widespread pain. Even more preferably the treatment results in at least a 90% reduction in said widespread pain. In such a case it is also preferred that treatment over a duration of 72 hours results in at least a 50% reduction in said widespread pain.

In addition, where the compound, pharmaceutically acceptable salt, prodrug or hydrate is for use in the treatment of fibromyalgia, the treatment may further result in a diminution of one or more symptoms selected from: tender point pain and tenderness, depression, dizziness, impaired concentration, irritable bowel syndrome, headache, fatigue, and sleep disturbance. Alternatively or in addition, the treatment may further results in an improvement of at least 50% of one or more symptoms selected from: trigger point pain and tenderness, depression, dizziness, impaired concentration, irritable bowel syndrome, headache, fatigue, and sleep disturbance.

Where the compound, pharmaceutically acceptable salt, prodrug or hydrate is for use in the treatment of multiple chemical sensitivity, the subject to be treated may suffer from an ill feeling or one or more symptoms that occur reproducibly in two or more organs in response to low levels of exposure to at least two unrelated chemicals, wherein the ill feeling or the one or more symptoms improve or resolve when the chemicals are removed. The one or more symptoms may be selected from fatigue, difficulty in concentrating, depression, memory loss, weakness, dizziness, headache, heat intolerance, and arthralgia. The at least two unrelated chemicals may be selected from aerosol air freshener, aerosol deodorant, after shave, asphalt pavement, cigar smoke, cigarette smoke, cologne, diesel exhaust, diesel fuel, dry cleaning fluid, floor cleaner, furniture polish, garage fumes, gasoline exhaust, hair spray, inset repellent, insecticide spray, laundry detergent, marking pen, nail polish, nail polish remover, oil-based paint, perfume in cosmetics, restroom deodorizer, shampoo, tar fumes from roof or road, tile cleaner, varnish, shellac and lacquer.

A compound, pharmaceutically acceptable salt, prodrug or hydrate of the first aspect of the present invention may be for use in the treatment or prophylaxis of widespread pain of at least three anatomical sites of a mammalian subject's body, wherein the compound, pharmaceutically acceptable salt, prodrug or hydrate is further suitable for the treatment or prophylaxis of one or more symptoms selected from trigger point tenderness, fatigue, irritable bowel syndrome, sleep disorder, chronic headache, jaw pain, cognitive impairment, memory impairment, post-exertional malaise, muscle pain, morning stiffness, menstrual cramping, numbness, tingling sensation, dizziness, and chemical sensitivity.

Alternatively, a compound, pharmaceutically acceptable salt, prodrug or hydrate of the first aspect of the present invention may be for use in the treatment or prophylaxis of the symptom of unexplained fatigue which is persisting or relapsing, wherein the compound, pharmaceutically acceptable salt, prodrug or hydrate is further suitable for the treatment or prophylaxis of one or more of the symptoms selected from impaired memory, impaired concentration, tender cervical or axillary lymph nodes in the neck region, sore throat, muscle pain, multi-joint pain, headache, unrefreshing sleep, and post-exertional malaise.

Alternatively still, a compound, pharmaceutically acceptable salt, prodrug or hydrate of the first aspect of the present invention may be for use in the treatment or prophylaxis of the symptom of pain in one or more trigger points, wherein the compound, pharmaceutically acceptable salt, prodrug or hydrate is further suitable for the treatment or prophylaxis of one or more of the symptoms selected from numbness, dizziness, headache, concentration impairment, memory impairment, sleep disorder, fluid retention, balance problems, and stiffness.

A second aspect of the present invention relates to composition comprising a compound, pharmaceutically acceptable salt, prodrug or hydrate of the first aspect of the present invention, and a pharmaceutically acceptable excipient.

The compound, pharmaceutically acceptable salt, prodrug or hydrate of the first aspect of the present invention, or the composition of the second aspect of the present invention, may be for oral or parenteral administration. Preferably they are for oral administration.

A third aspect of the present invention relates to a method for the treatment or prophylaxis of
(i) fibromyalgia;
(ii) chronic fatigue syndrome;
(iii) myofascial pain syndrome;
(iv) Gulf War syndrome;
(v) multiple chemical sensitivity;
(vi) widespread pain of at least three anatomical sites of a mammalian subject's body;
(vii) the symptom of unexplained fatigue which is persisting or relapsing;
(viii) the symptom of pain in one or more trigger points; and/or
(ix) one or more symptoms, in a subject that is a veteran of the Gulf War, selected from the group consisting of aching muscles, spasm, fatigue, irritability, thick saliva, weight loss, diarrhoea, skin rash, memory loss, dizziness, peripheral numbness, sleep disturbance, chronic fever, laboured breathing, and headache;
   said method comprising administering to a mammalian subject a therapeutically or prophylactically effective amount of a compound, or a pharmaceutically acceptable salt, prodrug or hydrate thereof, wherein the compound, pharmaceutically acceptable salt, prodrug or hydrate is a fatty acid oxidation inhibitor and/or a carbohydrate oxidation activator.

In a preferred embodiment of the third aspect of the present invention, the fatty acid oxidation inhibitor is a mitochondrial fatty acid oxidation inhibitor, more preferably a beta-oxidation inhibitor, such as an inhibitor of acyl-CoA dehydrogenase, enoyl-CoA hydratase, 3-hydroxyacyl-CoA dehydrogenase or 3-ketoacyl-CoA thiolase. Most preferably the beta-oxidation inhibitor is an inhibitor of 3-ketoacyl-CoA thiolase.

In an alternative embodiment of the third aspect of the present invention, the fatty acid oxidation inhibitor is a mitochondrial fatty acid transport inhibitor, such as an inhibitor of CPT-1, CPT-2, carnitine-acylcarnitine translocase or γ-butyrobetaine hydroxylase.

In a further embodiment of the third aspect of the present invention, the carbohydrate oxidation activator is a glucose oxidation activator, such as a glycolysis activator, a pyruvate dehydrogenase activator or a pyruvate dehydrogenase kinase inhibitor.

In any embodiment of the third aspect of the present invention, the compound, pharmaceutically acceptable salt, prodrug or hydrate may be selected from hypoglycin, 3-chloro-3-butenoylpantetheine, 3-pentenoylpantetheine, iodoacetamide, N-ethylmaleimide, dithioerythritol, EDTA, o-phenanthroline, 2-mercaptoacetate, iodoacetic acid, 1-ethyl-3-(3-dimethylaminopropyl)-carbodiimide, diethyldicarbonate, iodoacetate, salicylic acid, 5,5′-dithiobis(2-nitrobenzoic acid), N-bromosuccinimide, 4-pentenoic acid, 2-bromooctanoic acid, 4-bromocrotonic acid, 4-bromotiglic acid, trimetazidine, ranolazine, 4-bromo-2-octenoic acid, N-methylmaleimide, semicarbazide, tris(hydroxymethyl)aminomethane, benzotript, etomoxir, oxfenicine, 4-hydroxyphenylglyoxylate, 2-tetradecylglycidic acid, trimetazidine derivative S-15176, metoprolol, perhexiline, amiodarone, aminocarnitine, 11-trimethylamino-undecanoyl-DL-carnitine, cardiolipin, carnitine, chenodeoxycholic acid, cholic acid, deoxycarnitine, digitonin, ethyl 2-[6-(4-nitrophenoxy)hexyl]oxirane-2-carboxylate, γ-linolenic acid, hemipalmitoylcarnitinium bromide, L-palmitoylcarnitine, L-sulfocarnitine, octyl glucoside, palmitoylcholine, phosphatidylcholine, 2-(2-naphthalen-2-yloxyethoxy)thiophene-5-glyoxylic acid, thiolcarnitine, 2-(3-methyl-cinnamyl-hydrazono)propionate, 2-(3-phenylpropoxyimino)butyric acid, medium and long-chain acyl carnitines, BM 13.907, 3-(2,2,2-trimethylhydrazine)propionate, 2-oxoglutarate, 3,4-dihydroxybenzoate, 3-(2,2-dimethylcyclopropyl)propionic acid, 3-bromo-2-oxoglutarate, 3-glutathione-2-oxoglutarate, 3-trimethylaminopropyl-1-sulfonate, 2,2′-bipyridine, ascorbate, dioxane, riboflavin-5′-phosphate, iodosobenzoate, pyridine-2,4-dicarboxylate, quinacrine, succinic semialdehyde, dichloroacetate, 3,3,3-trifluoro-2-hydroxy-2-methylpropionamide, 2-chloroisohexanoate, 2-oxobutyrate, dichloroacetophenone, dihydrolipoic acid, 3,3-dichloro-2-benzofuran-1-one, pyruvamide, lipoic acid, rapamycin, thiamine diphosphate, dobutamine, or a pharmaceutically acceptable salt, prodrug or hydrate thereof. Preferably the compound, pharmaceutically acceptable salt, prodrug or hydrate is selected from hypoglycin, salicylic acid, 4-pentenoic acid, 2-bromooctanoic acid, 4-bromocrotonic acid, 4-bromotiglic acid, trimetazidine, ranolazine, etomoxir, oxfenicine, 4-hydroxyphenylglyoxylate, 2-tetradecylglycidic acid, trimetazidine derivative S-15176, metoprolol, perhexiline, amiodarone, aminocarnitine, 2-(3-methyl-cinnamyl-hydrazono)propionate, 2-(3-phenylpropoxyimino)butyric acid, medium and long-chain acyl carnitines, BM 13.907, 3-(2,2,2-trimethylhydrazine)propionate, dichloroacetate, or a pharmaceutically acceptable salt, prodrug or hydrate thereof. More preferably the compound, pharmaceutically acceptable salt, prodrug or hydrate is selected from 4-pentenoic acid, 2-bromooctanoic acid, 4-bromocrotonic acid, 4-bromotiglic acid, trimetazidine, ranolazine, or a pharmaceutically acceptable salt, prodrug or hydrate thereof. More preferably the compound, pharmaceutically acceptable salt, prodrug or hydrate is selected from trimetazidine, ranolazine, or a pharmaceutically acceptable salt, prodrug or hydrate thereof. In one embodiment of the third aspect of the present invention the compound, pharmaceutically acceptable salt, prodrug or hydrate is trimetazidine, in another embodiment the compound, pharmaceutically acceptable salt, prodrug or hydrate is a fatty acid oxidation inhibitor and/or a carbohydrate oxidation activator other than trimetazidine.

The method of the third aspect of the present invention may be for the treatment of
(i) fibromyalgia;
(ii) chronic fatigue syndrome;
(iii) myofascial pain syndrome;
(iv) Gulf War syndrome; and/or
(v) multiple chemical sensitivity;
   wherein the compound, pharmaceutically acceptable salt, prodrug or hydrate is administered over a duration of time effective to result in a diminution of one or more major symptoms associated with said condition. Preferably said administering is over a duration of time effective to result in at least a 50% improvement in said one or more major symptoms. Even more preferably said administering is over a duration of time effective to result in at least a 90% improvement in said one or more major symptoms.

Where the method is for the treatment of fibromyalgia, one of said major symptoms may be widespread pain of at least three anatomical sites of the subject's body. In such a case it is preferred that said administering is over a duration of time effective to result in at least a 50% reduction in said widespread pain. Even more preferably said administering is over a duration of time effective to result in at least a 90% reduction in said widespread pain. In such a case it is also preferred that said administering over a duration of 72 hours is effective to result in at least a 50% reduction in said widespread pain.

In addition, where the method is for the treatment of fibromyalgia, said administering may be further effective to result in a diminution of one or more symptoms selected from: tender point pain, and tenderness depression, dizziness, impaired concentration, irritable bowel syndrome, headache, fatigue, and sleep disturbance. Alternatively, or in addition, said administering may be further effective to result in an improvement of at least 50% of one or more symptoms selected from: trigger point pain and tenderness, depression, dizziness, impaired concentration, irritable bowel syndrome, headache, fatigue, and sleep disturbance.

Where the method is for the treatment of multiple chemical sensitivity, the subject to be treated may suffer from an ill feeling or one or more symptoms that occur reproducibly in two or more organs in response to low levels of exposure to at least two unrelated chemicals, wherein the ill feeling or the one or more symptoms improve or resolve when the chemicals are removed. The one or more symptoms may be selected from fatigue, difficulty in concentrating, depression, memory loss, weakness, dizziness, headache, heat intolerance, and arthralgia. The at least two unrelated chemicals may be selected from aerosol air freshener, aerosol deodorant, after shave, asphalt pavement, cigar smoke, cigarette smoke, cologne, diesel exhaust, diesel fuel, dry cleaning fluid, floor cleaner, furniture polish, garage fumes, gasoline exhaust, hair spray, inset repellent, insecticide spray, laundry detergent, marking pen, nail polish, nail polish remover, oil-based paint, perfume in cosmetics, restroom deodorizer, shampoo, tar fumes from roof or road, tile cleaner, varnish, shellac and lacquer.

A method of the third aspect of the present invention may be for the treatment or prophylaxis of widespread pain of at least three anatomical sites of a mammalian subject's body, wherein the method is further suitable for the treatment or prophylaxis of one or more symptoms selected from trigger point tenderness, fatigue, irritable bowel syndrome, sleep disorder, chronic headache, jaw pain, cognitive impairment, memory impairment, post-exertional malaise, muscle pain, morning stiffness, menstrual cramping, numbness, tingling sensation, dizziness, and chemical sensitivity.

Alternatively, a method of the third aspect of the present invention may be for the treatment or prophylaxis of the symptom of unexplained fatigue which is persisting or relapsing, wherein the method is further suitable for the treatment or prophylaxis of one or more of the symptoms selected from impaired memory, impaired concentration, tender cervical or axillary lymph nodes in the neck region, sore throat, muscle pain, multi-joint pain, headache, unrefreshing sleep, and post-exertional malaise.

Alternatively still, a method of the third aspect of the present invention may be for the treatment or prophylaxis of the symptom of pain in one or more trigger points, wherein the method is further suitable for the treatment or prophylaxis of one or more of the symptoms selected from numbness, dizziness, headache, concentration impairment, memory impairment, sleep disorder, fluid retention, balance problems, and stiffness.

In any embodiment of the third aspect of the present invention, said administering may be by oral or parenteral administration. Preferably said administering is by oral administration.

A fourth aspect of the present invention relates to a kit comprising a compound, or a pharmaceutically acceptable salt, prodrug or hydrate thereof, in packaged form, and instructions for administering said compound, pharmaceutically acceptable salt, prodrug or hydrate for the treatment or prophylaxis of
(i) fibromyalgia;
(ii) chronic fatigue syndrome;
(iii) myofascial pain syndrome;
(iv) Gulf War syndrome;
(v) multiple chemical sensitivity;
(vi) widespread pain of at least three anatomical sites of a mammalian subject's body;
(vii) the symptom of unexplained fatigue which is persisting or relapsing;
(viii) the symptom of pain in one or more trigger points; and/or
(ix) one or more symptoms, in a subject that is a veteran of the Gulf War, selected from the group consisting of aching muscles, spasm, fatigue, irritability, thick saliva, weight loss, diarrhoea, skin rash, memory loss, dizziness, peripheral numbness, sleep disturbance, chronic fever, laboured breathing, and headache;
   wherein the compound, pharmaceutically acceptable salt, prodrug or hydrate is a fatty acid oxidation inhibitor and/or a carbohydrate oxidation activator. Preferably said compound, pharmaceutically acceptable salt, prodrug or hydrate is in the form of a tablet, syrup, suspension, and capsule.

In a preferred embodiment of the fourth aspect of the present invention, the fatty acid oxidation inhibitor is a mitochondrial fatty acid oxidation inhibitor, more preferably a beta-oxidation inhibitor, such as an inhibitor of acyl-CoA dehydrogenase, enoyl-CoA hydratase, 3-hydroxyacyl-CoA dehydrogenase or 3-ketoacyl-CoA thiolase. Most preferably the beta-oxidation inhibitor is an inhibitor of 3-ketoacyl-CoA thiolase.

In an alternative embodiment of the fourth aspect of the present invention, the fatty acid oxidation inhibitor is a mitochondrial fatty acid transport inhibitor, such as an inhibitor of CPT-1, CPT-2, carnitine-acylcarnitine translocase or γ-butyrobetaine hydroxylase.

In a further embodiment of the fourth aspect of the present invention, the carbohydrate oxidation activator is a glucose oxidation activator, such as a glycolysis activator, a pyruvate dehydrogenase activator or a pyruvate dehydrogenase kinase inhibitor.

In any embodiment of the fourth aspect of the present invention, the compound, pharmaceutically acceptable salt, prodrug or hydrate may be selected from hypoglycin, 3-chloro-3-butenoylpantetheine, 3-pentenoylpantetheine, iodoacetamide, N-ethylmaleimide, dithioerythritol, EDTA, o-phenanthroline, 2-mercaptoacetate, iodoacetic acid, 1-ethyl-3-(3-dimethylaminopropyl)-carbodiimide, diethyldicarbonate, iodoacetate, salicylic acid, 5,5′-dithiobis(2-nitrobenzoic acid), N-bromosuccinimide, 4-pentenoic acid, 2-bromooctanoic acid, 4-bromocrotonic acid, 4-bromotiglic acid, trimetazidine, ranolazine, 4-bromo-2-octenoic acid, N-methylmaleimide, semicarbazide, tris(hydroxymethyl)aminomethane, benzotript, etomoxir, oxfenicine, 4-hydroxyphenylglyoxylate, 2-tetradecylglycidic acid, trimetazidine derivative S-15176, metoprolol, perhexiline, amiodarone, aminocarnitine, 11-trimethylamino-undecanoyl-DL-carnitine, cardiolipin, carnitine, chenodeoxycholic acid, cholic acid, deoxycarnitine, digitonin, ethyl 2-[6-(4-nitrophenoxy)hexyl]oxirane-2-carboxylate, γ-linolenic acid, hemipalmitoylcarnitinium bromide, L-palmitoylcarnitine, L-sulfocarnitine, octyl glucoside, palmitoylcholine, phosphatidylcholine, 2-(2-naphthalen-2-yloxyethoxy)thiophene-5-glyoxylic acid, thiolcarnitine, 2-(3-methyl-cinnamyl-hydrazono)propionate, 2-(3-phenylpropoxyimino)butyric acid, medium and long-chain acyl carnitines, BM 13.907, 3-(2,2,2-trimethylhydrazine)propionate, 2-oxoglutarate, 3,4-dihydroxybenzoate, 3-(2,2-dimethylcyclopropyl)propionic acid, 3-bromo-2-oxoglutarate, 3-glutathione-2-oxoglutarate, 3-trimethylaminopropyl-1-sulfonate, 2,2′-bipyridine, ascorbate, dioxane, riboflavin-5′-phosphate, iodosobenzoate, pyridine-2,4-dicarboxylate, quinacrine, succinic semialdehyde, dichloroacetate, 3,3,3-trifluoro-2-hydroxy-2-methylpropionamide, 2-chloroisohexanoate, 2-oxobutyrate, dichloroacetophenone, dihydrolipoic acid, 3,3-dichloro-2-benzofuran-1-one, pyruvamide, lipoic acid, rapamycin, thiamine diphosphate, dobutamine, or a pharmaceutically acceptable salt, prodrug or hydrate thereof. Preferably the compound, pharmaceutically acceptable salt, prodrug or hydrate is selected from hypoglycin, salicylic acid, 4-pentenoic acid, 2-bromooctanoic acid, 4-bromocrotonic acid, 4-bromotiglic acid, trimetazidine, ranolazine, etomoxir, oxfenicine, 4-hydroxyphenylglyoxylate, 2-tetradecylglycidic acid, trimetazidine derivative S-15176, metoprolol, perhexiline, amiodarone, aminocarnitine, 2-(3-methyl-cinnamyl-hydrazono)propionate, 2-(3-phenylpropoxyimino)butyric acid, medium and long-chain acyl carnitines, BM 13.907, 3-(2,2,2-trimethylhydrazine)propionate, dichloroacetate, or a pharmaceutically acceptable salt, prodrug or hydrate thereof. More preferably the compound, pharmaceutically acceptable salt, prodrug or hydrate is selected from 4-pentenoic acid, 2-bromooctanoic acid, 4-bromocrotonic acid, 4-bromotiglic acid, trimetazidine, ranolazine, or a pharmaceutically acceptable salt, prodrug or hydrate thereof. More preferably the compound, pharmaceutically acceptable salt, prodrug or hydrate is selected from trimetazidine, ranolazine, or a pharmaceutically acceptable salt, prodrug or hydrate thereof. In one embodiment of the fourth aspect of the present invention the compound, pharmaceutically acceptable salt, prodrug or hydrate is trimetazidine, in another embodiment the compound, pharmaceutically acceptable salt, prodrug or hydrate is a fatty acid oxidation inhibitor and/or a carbohydrate oxidation activator other than trimetazidine.

Each of the herein-described features of the invention is meant to apply equally to each and every embodiment as described herein, unless otherwise indicated.

Additional objects, advantages and novel features of the invention will be set forth in the description that follows, and in part, will become apparent to those skilled in the art upon reading the following, or may be learned by practice of the invention.

### DETAILED DESCRIPTION OF THE INVENTION

### Definitions

Before describing the present invention in detail, it is to be understood that this invention is not limited to particular formulations, administration modes, and the like, as such may vary, as will be apparent from the accompanying description.

It must be noted that, as used in this specification and the intended claims, the singular forms “a”, “an” and “the” include plural referents unless the context clearly dictates otherwise. Thus, for example, reference to “a drug” includes a single drug as well as two or more of the same or different drugs, reference to “an optional excipient” refers to a single optional excipient as well as two or more of the same or different optional excipients, and the like.

In describing and claiming the present invention, the following terminology will be used in accordance with the definitions described below.

“Pharmaceutically acceptable excipient or carrier” refers to an excipient that may optionally be included in the compositions of the invention and that causes no significant adverse toxicological effects to the patient.

“Pharmacologically effective amount”, “physiologically effective amount” and “therapeutically effective amount” are used interchangeably herein to mean the amount of a drug or drug-combination that is needed to provide a desired level of drug in the bloodstream or in the target tissue. The precise amount will depend upon numerous factors, e.g., the particular drug or drugs employed, the components and physical characteristics of the therapeutic composition, intended patient population, individual patient considerations, and the like, and can readily be determined by one skilled in the art, based upon the information provided herein.

“Pharmaceutically acceptable salt” includes, but is not limited to, amino acid salts, salts prepared with inorganic acids, such as chloride, sulfate, phosphate, diphosphate, hydrobromide, hydrochloride, and nitrate salts, or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium). In a preferred embodiment, trimetazidine is provided as a hydrochloride or dihydrochloride salt, and ranolazine is provided as a dihydrochloride salt.

“Prodrug” refers to any derivative of a drug that is metabolized or otherwise converted into an active form upon introduction into the body of an animal. Prodrugs are well known to those skilled in the art of pharmaceutical chemistry, and provide benefits such as increased absorption and halflife. Prodrugs of this invention may be formed when, for example, hydroxy groups are esterified or alkylated, or when carboxyl groups are esterified. Those skilled in the art of drug delivery will readily appreciate that the pharmacokinetic properties of the compounds of the invention may be controlled by an appropriate choice of moieties to produce prodrug derivatives.

“Active molecule” or “active agent” as described herein includes any agent, drug, compound, composition of matter or mixture which provides some pharmacologic, often beneficial, effect that can be demonstrated in vivo or in vitro. This includes foods, food supplements, microbiologicals, nutrients, nutriceuticals, drugs, vaccines, antibodies, vitamins, and other beneficial agents. As used herein, the terms further include any physiologically or pharmacologically active substance that produces a localized or systemic effect in a patient.

“Substantially” or “essentially” means nearly totally or completely, for instance, 95% or greater of some given quantity. For example, a substantial elimination of one or more symptoms or clinical indicators, e.g., of fibromyalgia syndrome or chronic fatigue syndrome, etc., means a reduction in severity of 95% or more of a symptom such as widespread pain, fatigue, irritable bowel syndrome, insomnia, cognitive and memory impairment, morning stiffness, dizziness, irritability, depression, as assessed by any clinically acceptable method, or an improvement of at least 95% of a given clinical indicator.

A “diminution” of one or more symptoms or clinical indicators, e.g., a major symptom associated with fibromyalgia syndrome, chronic fatigue syndrome, myofascial pain syndrome, or any other condition treatable in accordance with the present invention, means a measurable reduction in the severity of such one or more symptoms, as assessed by any clinically acceptable method, or a measurable improvement of a given clinical indicator, as assessed by a skilled clinician.

The terms “patient” and “subject” are used interchangeably and refer to a living mammalian organism suffering from or prone to a condition that can be prevented or treated by administration of a drug or combination of drugs of the invention, and includes both humans and animals.

“Optional” or “optionally” means that the subsequently described circumstance may or may not occur, so that the description includes instances where the circumstance occurs and instances where it does not.

The term “treating” includes preventing, essentially eradicating, or ameliorating one or more major symptoms associated with the condition being treated, e.g., FMS, CFS, MFS, and the like. Thus, treatment may be accomplished, for example, when the patient reports decreased severity, duration, or recurrence of pain, a reduction in the number of anatomical sites affected by pain, a reduction in the number of tender points or trigger points, etc.

“Widespread pain” occurs when all of the following are present: axial skeletal pain, pain on the left hand side of the body, pain on the right hand side of the body, pain above the waist and pain below the waist (see Arthritis Rheum, 1990, 33: 160-172 (Medline)).

“Mitochondrial fatty acid oxidation” refers to the oxidation of fatty acids that occurs within the mitochondria. A “mitochondrial fatty acid oxidation inhibitor” is a substance that inhibits the oxidation of fatty acids, said inhibition occurring within the mitochondria. “Beta oxidation” refers to a particular mitochondrial fatty acid oxidation pathway in which fatty acids are broken down into acetyl-coenzyme A by repeated oxidation at the beta-carbon atom. The term “beta oxidation inhibitor” accordingly refers to a substance that inhibits the beta oxidation of fatty acids, said inhibition occurring within the mitochondria.

“Mitochondrial fatty acid transport” refers to the transfer of fatty acids into the mitochondria through the mitochondrial membranes; the term “mitochondrial fatty acid transport inhibitor” accordingly refers to a substance that inhibits mitochondrial fatty acid transport.

“Carbohydrate oxidation activator” refers to a substance that increases the oxidation of carbohydrates within the body. Preferably the carbohydrate oxidation activator increases the oxidation of monosaccharides within the body. For instance, the carbohydrate oxidation activator may increase the rate of glycolysis (the breakdown of glucose into pyruvate), and/or may stimulate the post-glycolysis process, for example, by activating pyruvate dehydrogenase or by inhibiting pyruvate dehydrogenase kinase.

“Short chain fatty acid” refers to an optionally substituted, saturated or unsaturated, straight-chained, branched or cyclic carboxylic acid comprising 2 to 6 carbon atoms, such as acetic acid, propionic acid, isobutyric acid, butyric acid, isovaleric acid, valeric acid, caproic acid, lactic acid and succinic acid.

“Medium chain fatty acid” refers to an optionally substituted, saturated or unsaturated, straight-chained, branched or cyclic carboxylic acid comprising 7 to 12 carbon atoms, such as caprylic add, capric acid, lauric add, lauroleic acid, heptanoic acid, nonanoic acid and undecanoic acid.

“Long chain fatty acid” refers to an optionally substituted, saturated or unsaturated, straight-chained, branche, or cyclic carboxylic acid comprising 13 or more carbon atoms, such as myristic acid, myristoleic acid, pentadecanoic acid, palmitic acid, palmitoleic acid, margaric acid, stearic acid, dihydroxystearic acid, oleic acid, ricinoleic acid, elaidic acid, linoleic acid, eleostearic acid, licanic acid, arachidonic acid, arachidic acid, eicosenoic acid, eicosapentaenoic acid, behenic acid, erucic acid, docosahexaenoic acid and lignoceric acid.

### Method of Treatment—Overview

As described previously, the inventors have discovered that fatty acid oxidation inhibitors and/or carbohydrate oxidation activators are uniquely effective in treating the symptoms of FMS, and in treating the widespread spectrum of FMS-associated symptomatology (e.g., CFS, MPS, MCS (multiple chemical sensitivities), and the like).

In each of the instances in which the applicants have administered a fatty acid oxidation inhibitor/carbohydrate oxidation activator (e.g., trimetazidine) to a patient suffering from FMS, and experiencing one or more of the following symptoms: widespread pain, tender points, fatigue, irritable bowel syndrome, insomnia, cognitive and memory impairment, morning stiffness, dizziness, depression, acid reflux, following a course of treatment of at least four weeks, the patient has reported at least a remarkable and measurable reduction in widespread pain, and in some instances, has described substantially complete alleviation of widespread pain, if not of all of the symptoms. See, for example, Examples 1-4 herein, which demonstrate a significant FMS symptom-modifying response upon administration of trimetazidine.

Briefly, in studies of eleven human subjects, each reported a remarkable improvement in widespread pain experienced by the subjects prior to treatment. In sum, within only 72 hours of commencement of treatment with a fatty acid oxidation inhibitor/carbohydrate oxidation activator, each of the subjects reported a marked reduction in widespread pain, and where applicable, tender point pain. More specifically, each of the subjects treated in accordance with the invention reported, on average, a 90% reduction in widespread pain within 72 hours of commencing treatment. Further, all patients reported significant improvement of minor symptoms within the same period. Follow-up examinations at either four months or four weeks of treatment revealed, for all patients, remarkably sustained relief or even improved relief of widespread pain, e.g., 90-95% improvement. Thus, it can be seen that the method of the present invention is remarkably effective in the treatment of rheumatic conditions such as FMS and the like.

### Method of Diagnosis

Fibromyalgia. Fibromyalgia, also referred to as fibromyalgia syndrome, is part of a spectrum of chronic widespread pain of unknown origin (Bennett R, Curr Opin Rheumatol, March 1998, 10(2): 95-103). The terms “fibromyalgia” and “fibromyalgia syndrome” are used interchangeably herein. According to Bennett (ibid), the prevalence of chronic widespread pain is several times higher than fibromyalgia as defined by the 1990 American College of Rheumatology guidelines. Thus, the method of the present invention is directed to treating not only fibromyalgia, but also to treating the spectrum of chronic widespread pain having fibromyalgia-associated symptomatology, e.g., CFS, MPS, MCS, and the like. Although pain and tenderness are its defining features, fatigue, sleep disturbance, depression, and poor concentration are also common.

Fibromyalgia can be diagnosed in a clinical setting by counting the number of tender points a patient has (Wolfe F, Ann Rheum Dis, April 1997, 56: 268-272). In 1990, the American College of Rheumatology established criteria for diagnosing fibromyalgia, which includes the presence of 11 or more tender points and widespread pain of at least three months' duration (Wolfe F, et al., The American College of Rheumatology, 1990 Criteria for the Classification of Fibromyalgia: Report of the Multicenter Criteria Committee, Arthritis Rheum, 1990, 33: 160-172 (Medline)). Such pain is typically present in all four quadrants of the body, i.e., on both the left and right side of the body and above and below the waist.

In assessing a subject for FMS, the patient is typically examined by undergoing a count of tender points using the 18 specified sites specified in the American College of Rheumatology 1990 Classification Criteria, ACRCC (ibid). Tender point data are reported as a count of positive test sites. Namely, the 18 tender point sites are at the occiput (bilateral, at the suboccipital muscle insertions), the low cervical (bilateral, at the anterior aspects of the intertransverse spaces at C5-C7), the trapezius (bilateral, at the midpoint of the upper border), the supraspinatus (bilateral, at origins, above the scapula spine near the medial border), the second rib (bilateral, at the second costochondral junctions, just lateral to the junctions on upper surfaces), the lateral epicondyle (bilateral, 2 cm distal to the epicondyles), the gluteal (bilateral, in upper outer quadrants of buttocks in anterior fold of muscle), the greater trochanter (bilateral, posterior to the trochanteric prominence) and the knee (bilateral, at the medial fat pad proximal to the joint line).

Pain threshold may be assessed using a dolorimetry examination (a pressure algometer). Such an examination is performed at the trapezii, knees, lateral epicondyle, and second rib. The examiner places the rubber tip of the dolorimeter on the examination site and gradually increases the pressure (e.g., at a rate of approximately 1 kg/cm² per second). The patient is asked to report the moment when the sensation at the site changes from that of pressure to that of pain. The force is then recorded at that point. The overall dolorimetry score is the mean of the sites examined. Among persons with FMS in a population survey, mean dolorimetry scores were approximately 2.7 kg/cm² (Wolfe F, et al., Arthritis Rheum, 1995, 38: 19-28), while a median value in the normal population for women is 4.25 kg/cm² and 6.0 kg/cm² for men (Wolfe F, et al., J Rheumatol, 1995, 22: 151-156).

In evaluating a subject for FMS, a questionnaire is also typically used. An exemplary questionnaire is The Clinical Health Assessment Questionnaire (CLINHAQ) (Wolfe F, Rheumatoid Arthritis: Pathogenesis, Assessment, Outcome, and Treatment, New York, Marcel Dekker, 1994, 463-514). This questionnaire contains self reports for the Health Assessment Questionnaire (HAQ) disability index (Fries J F, et al., Arthritis Rheum, 1980, 23: 137-145), arthritis impact measurement scales (AIMS) anxiety and depression index (Hawley D J, et al., J Rheumatol, 1993, 20: 2025-203), visual analogue scale (VAS) pain, VAS global severity, VAS gastrointestinal symptoms, VAS sleep problems, VAS fatigue, satisfaction with health and patient estimate of health status. In 1996, the helplessness subscale of the rheumatology attitudes index (RAI) was added to the CLINHAQ (deVellis R F, et al., J Rheumatol, 1993, 20: 866-869). The variables contained in this questionnaire consider factors that are thought to be of major importance in fibromyalgia (Burckhardt C S, et al., J Rheumatol, 1991, 18: 728-733; Simms R W, et al., J Rheumatol, 1991, 18: 1558-1563).

Since there are no definite etiologic markers used for the diagnosis of FMS, approaches such as the above are typically used in diagnosing a patient with FMS, along with laboratory tests used to exclude the possibility of other disorders that could cause similar symptoms.

A study by Wolfe (Wolfe F, Ann Rheum Dis, 1997, 56: 268-271), reports that FMS tenderness and symptoms are part of a continuum, and that, in a broad sense outside of a clinical setting, there is no discrete point where FMS does or does not exist, and that it is important to recognize the distress symptoms—whether or not the patient reaches the fibromyalgia diagnostic threshold as set forth in the ACRCC. For instance, alternative less stringent criteria for a positive diagnosis of FMS include, as part of the major criteria, generalized pain or stiffness of at least three anatomical sites for at least three months, and the existence of six or more tender points.

Thus, in accordance with the above, fatty acid oxidation inhibitors and/or carbohydrate oxidation activators such as trimetazidine are useful in the treatment of FMS and of syndromes similar to fibromyalgia. That is to say, in one aspect, the invention encompasses the administering of a fatty acid oxidation inhibitor and/or a carbohydrate oxidation activator for the treatment FMS, where the FMS patient is one that has experienced as major symptoms associated with FMS: (i) widespread pain associated with at least three anatomical sites of a duration of at least three months, and (ii) five or more tender points rather than the absolute 11 or more tender points defined by the ACRCC, even if such widespread pain does not fall within the clinical diagnosis of FMS. That is to say, treatment for FMS in accordance with the present invention includes treatment of subjects having a history of widespread pain of at least three months' duration and having fewer symptoms or tender points than 11, e.g., the patient may have 5 tender points, or 6 tender points, or 7 tender points, or 8 tender points, or 9 tender points, or even 10 tender points, or 11 tender points or more. Minor symptoms, which may also be treated in accordance with the invention, i.e., prevented, ameliorated, or eradicated, include fatigue, irritable bowel syndrome, sleep disorder, chronic headaches, jaw pain, cognitive or memory impairment, post-exertional malaise and muscle pain, morning stiffness, menstrual cramping, numbness and tingling sensations, dizziness, and skin and chemical sensitivities.

Treatment of any one or more of the major or minor symptoms described above, for this and related rheumatic conditions described herein, includes an identifiable diminution or amelioration of one or more symptoms or clinical indicators associated with fibromyalgia syndrome, chronic fatigue syndrome, myofascial pain syndrome, or any other condition treatable in accordance with the present invention, i.e., a measurable reduction in the severity of such one or more symptoms, as assessed by any clinically acceptable method, or a measurable improvement of a given clinical indicator, e.g., as assessed by a skilled clinician. Treatment with a fatty acid oxidation inhibitor and/or a carbohydrate oxidation activator is therefore effective to result in a measurable improvement in one or more symptoms associated with the subject rheumatic condition. Thus, treatment with a fatty acid oxidation inhibitor and/or a carbohydrate oxidation activator may be effective to result in an improvement of at least about 20%, preferably at least about 30%, more preferably at least about 40%, even more preferably at least about 50%, and even more preferably at least about 60%, and even more preferably at least about 70%, and even more preferably at least about 80%, even more preferably at least about 90%, and most preferably at least about 95% or greater in a given major or minor symptom.

In one embodiment of the present invention, the treatment may be of primary fibromyalgia. In another embodiment, the treatment may be of secondary fibromyalgia. As used herein, the term “primary fibromyalgia” refers to fibromyalgia in which the only rheumatic disorder the patient is suffering from is fibromyalgia, whereas the term “secondary fibromyalgia” refers to fibromyalgia that occurs in conjunction with another diagnosed rheumatic disorder.

Chronic Fatigue Syndrome. Chronic fatigue syndrome is typically diagnosed using the Center for Disease Control and Prevention (CDC) 1994 guidelines published in the Annuals of Internal Medicine, 121, 12: 953-9. To meet these criteria, patients must have severe, unexplained fatigue that is not relieved by rest, which can cause disability and which has an identifiable onset. It should be persistent or relapsing fatigue that lasts for at least six or more consecutive months. In accordance with the present invention, persistent fatigue such as the above is considered a major symptom of CFS. According to the CDC guidelines, patients must also exhibit four or more of the following symptoms, considered herein as minor or secondary symptoms: impaired memory or concentration, tender cervical or axillary lymph nodes in the neck region, sore throat, muscle pain, multi-joint pain (but not arthritis), new onset headaches (of a new type, pattern or severity), unrefreshing sleep, and post-exertional malaise. As in the case of FMS, patients should be clinically evaluated to exclude other conditions that could be the cause of the above symptoms.

In many cases, FMS and CFS are co-existing conditions. It has been estimated that 20% to 70% of patients with FMS meet the criteria for CFS and that about 35% to 75% of patients with CFS also have FMS (Jason L, et al., Psychosomatic Medicine, 2000, 62: 655-63).

Illustrative measures for assessing CFS are as follows. The Fatigue Scale, as originally used in a hospital-based case study (Wessely S, et al., J Neurol Neurosurg Psychiatry, 1989, 52: 940-8) and further refined by Chalder et al. (J Psychosom Med, 1993, 37: 147-53), can be used to assess fatigue experienced by a subject. The fatigue scale is an eleven-item scale that has responses rated on a four-option continuum: the total score ranges from 0 to 33 (with a higher score indicating greater fatigue).

In combination with the above, one can optionally use a psychiatric interview such as the SCID (Structured Clinical Interview for DSM-IV Axis I Disorders, DSM-IV. Diagnostic and statistical manual of mental disorders, 4^{th} ed., Washington D.C., American Psychiatric Association, 1994).

Additionally, to assess CFS, one can administer a medical questionnaire such as The Chronic Fatigue Questionnaire (Komaroff A L, et al., Am J Med, 1996, 100: 56-64) to assess symptoms related to CFS, as well as rule out exclusionary medical conditions.

Also, a detailed physical examination is typically conducted to rule out other possible medical conditions, including an 18-tender point examination as described above. Laboratory tests typically include one or more of the following: chemical screen (glucose, calcium, electrolytes, uric acid, liver function parameters, and renal function parameters), complete blood count with differential and platelet counts, T4 and thyroid-stimulating hormone, erythrocyte sedimentation rate, arthritic profile (e.g., rheumatoid factor and antinuclear antibody), hepatitis B surface antigen, creatine phosphokinase, an HIV screen and urinalysis.

Myofascial Pain Syndrome. Myofascial pain syndrome (also known as regional pain syndrome) has no uniformly accepted definition but is characterized as a regional muscle pain syndrome accompanied by trigger points (TPs) and their associated reflexes. A trigger point is a hyperirritable spot within a taut band of skeletal muscle or muscle fascia which is painful on compression and gives rise to characteristic referral pain patterns, tenderness and autonomic phenomena. A trigger point may be active or latent. In accordance with the present invention, the existence of one or more trigger points is considered a major symptom of MPS.

Although there are no clear clinical criteria for diagnosing MPS, to diagnose an active myofascial trigger point, one looks for the following (Travell J F, Simons D G, Myofascial Pain and Dysfunction: The Trigger Point Manual. Baltimore, Williams & Wilkint, vol. 1, 1983, 18-19): (1) a history of sudden onset during or shortly following acute overload stress, or a history of gradual onset with chronic overload of the affected muscle; (2) characteristic patterns of pain that are referred from myofascial TPs; (3) weakness and restriction in the stretch range of motion of the affected muscle; (4) a taut, palpable band in the affected muscle; (5) exquisite, focal tenderness to digital pressure in the band of taut muscle fibers—the trigger point; (6) a local twitch response elicited through snapping palpation or needling of the tender spot or trigger point; (7) the reproduction of the patient's pain complaint by pressure on, or needling of, the tender spot or trigger point; and (8) the elimination of symptoms by therapy directed specifically to the affected muscles. Individuals typically will not meet all of the above criteria, however, the existence of one or more trigger points is a major symptom of all patients diagnosed with MPS.

A taut band (as mentioned in item (4) above) is a ropelike swelling found within the muscle, probably due to sustained shortening of muscle fibers. A twitch response (as mentioned in item (6) above) is a transient contraction of the muscle fibers of the taut band containing a TP. The twitch response can be elicited by snapping palpation of the trigger point, or more commonly by precise needling. A TP is typically identified by application of digital pressure or needling of the tender spot (typically by application of sustained pressure for 6-60 seconds), resulting in induction or reproduction of some or all of the patient's pain complaint.

Minor or secondary symptoms of MPS may include numbness, dizziness, headaches, concentration and memory problems, sleep disorders, fluid retention, balance problems, and stiffness.

As with the above syndromes, there is no single diagnostic test that confirms the diagnosis of MPS.

As used herein, MPS occurs where a subject possesses one or more trigger points. In one embodiment, the subject has two or more trigger points. In another embodiment, the subject has three or more trigger points. In yet another embodiment, the subject has four or more trigger points. In some embodiments, MPS occurs where a subject, in addition to possessing the number of trigger points defined above, also suffers from one or more minor or secondary symptoms as defined above.

Multiple Chemical Sensitivity (MCS) Syndrome. Multiple chemical sensitivity syndrome has been described under various names since the 1940s. A 1999 case definition of MCS describes it as a chronic condition with reproducible symptoms involving multiple organ systems whose symptoms are produced by low levels of exposure to multiple, chemically unrelated substances and improve or resolve upon removal of the chemical agents (Bartha L, et al., Arch Environ Health, 1999, 54: 147-9). Self-reported high levels of reactivity to chemical exposure have been found in patients with CFS in comparison to healthy control subjects (Friedberg F, et al., Proceedings of the American Association of Chronic Fatigue Syndrome Research Conference, October 1994, Fort Lauderdale (Fla.)). In a sample of 33 Gulf War veterans with CFS, 42% had concurrent MCS and 6% had FMS (Pollet C, et al., J Med, 1998, 29: 101-13). In another sample of 100 patients with MCS, it was found that 88% met criteria for CFS and 49% met the criteria for FMS (Donnay A, Ziem G, Proceedings of the American Association of Chronic Fatigue Syndrome Research Conference, October 1998, Cambridge (Mass.)).

A diagnosis of MCS typically involves screening as described above for the other related syndromes. For example, a clinician will typically gather a patient history, conduct a detailed physical examination as well as conduct laboratory tests to rule out exclusionary medical conditions. Additionally, a medical questionnaire is typically administered in which subjects are asked if they had symptoms of feeling ill from a low level of exposure to two or more listed chemical agents that affected two or more organ systems (Bartha L, 1999, ibid). Subjects are diagnosed with MCS if they report positively to the above inquiry. Thus, in accordance with the present invention, a major symptom of MCS is an ill feeling or symptoms that occur reproducibly in two or more organs in response to low levels of exposure to at least two unrelated chemicals that improves or resolves when the chemicals are removed. Symptoms typically occur in one of three categories: central nervous system symptoms, respiratory and mucosal irritation, or gastrointestinal problems, and include fatigue, difficulty concentrating, depression, memory loss, weakness, dizziness, headache, heat intolerance, and arthralgia. Exposures include aerosol air freshener, aerosol deodorant, after shave, asphalt pavement, cigar smoke, cigarette smoke, cologne, diesel exhaust, diesel fuel, dry cleaning fluid, floor cleaner, furniture polish, garage fumes, gasoline exhaust, hair spray, insect repellant, insecticide spray, laundry detergent, marking pens, nail polish, nail polish remover, oil-based paint, paint thinner, perfumes in cosmetics, restroom deodorizers, shampoo, tar fumes from roof or road, tile cleaners, varnish, shellac and lacquer (Lax M B, Henneberger P K, Arch Environ Health, 1995, 50: 425-31).

In one embodiment, the ill feeling or symptoms occur reproducibly in two or more organs in response to low levels of exposure to at least three unrelated chemicals, and improve or resolve when the chemicals are removed. In another embodiment, the ill feeling or symptoms occur in response to low levels of exposure to at least four, five or six unrelated chemicals, and improve or resolve when the chemicals are removed.

Gulf War Syndrome (GWS). Yet another condition treatable in accordance with the present invention is GWS. GWS is the name given to a variable combination of unexplainable psychological and physical complaints experienced by veterans of the Persian Gulf War. Symptoms may include aching muscles, spasms, fatigue, irritability, thick saliva, weight loss, diarrhea, skin rashes, memory loss, dizziness, peripheral numbness, sleep disturbance, along with chronic fevers, labored breathing, and headaches. There is currently no well-accepted explanation for the symptoms experienced by Gulf War veterans, although several theories exist including exposure to chemical warfare agents, psychological factors, and exposure to other chemicals such as depleted uranium. In accordance with the invention, a subject that is a veteran of the Gulf War and that has experienced one or more of the above symptoms, each of which is considered herein as a major symptom, is considered to have GWS.

In one embodiment, said subject experiences two or more of the above symptoms. In another embodiment, said subject experiences three or more, or four or more of the above symptoms.

A subject with GWS may be one who is experiencing GWS 1, which involves symptoms such as sleep and memory disturbance. A subject with GWS may also be one who is experiencing GWS 2, whose symptoms include confusion and dizziness. Yet another subject with GWS may be one experiencing GWS 3, whose symptoms include muscle and joint pain. The three distinct syndromes were identified by a medical team in the U.S. using a statistical technique called factor analysis, which reveals unusual clusters of symptoms (Kang H K, et al., Am J Epidemiol, 2003, 157: 141-8).

### Formulations

While limited success has been associated with prior therapies to date for FMS and related rheumatic conditions as described above, the inventors have discovered that administration of a formulation comprising a fatty acid oxidation inhibitor and/or a carbohydrate oxidation activator is surprisingly effective in treating symptoms associated with FMS. As can be seen in the supporting examples, patients suffering from symptoms such as widespread pain, trigger point pain and tenderness, depression, dizziness, impaired concentration, symptoms of irritable bowel syndrome, fatigue, headache, impaired memory, and sleep disturbance, among others, reported significant, if not remarkable and sustained improvement in their symptoms after a course of therapy with a fatty acid oxidation inhibitor/carbohydrate oxidation activator (e.g., trimetazidine).

Thus, one aspect of the present invention is directed to compositions, combinations, and methods comprising the use of a fatty acid oxidation inhibitor and/or a carbohydrate oxidation activator, as will now be described in further detail in the sections that follow.

Additional Active Agents: In addition to a fatty acid oxidation inhibitor and/or a carbohydrate oxidation activator, a therapeutic composition of the invention may optionally include a therapeutically effective amount of one or more additional active agents, herbs, vitamins, minerals, or other supplements useful in treating the subject rheumatic condition. If desired, in addition to a fatty acid oxidation inhibitor and/or a carbohydrate oxidation activator, a composition of the invention may, but does not necessarily, also include one or more of the following: antidepressants such as amitriptyline, duloxetine, fluoxetine, paroxetine, sertraline, venlafaxine, trazodone, and bupropion; muscle relaxants such as cyclobenzaprine; analgesics such as tramadol, naproxen, ibuprofen, and piroxicam; low dose tricyclic agents such as doxepin, desipramine, and nortriptyline; anxiolytic agents such as alprazolam, clonazepam, and lorazepam; antihistamines such as astemizole, and loratadine; antihypotensive agents such as fludrocortisone; and beta blockers such as atenolol, among others.

Alternatively, a therapeutically effective amount of any one or more of the above active agents may be co-administered, as a separate dosage form, with a fatty acid oxidation inhibitor and/or a carbohydrate oxidation activator for treatment of, e.g., FMS, CFS, MPS, or GWS.

Excipients/Additives: Optionally, the compositions of the invention may further comprise one or more pharmaceutically acceptable excipients to provide a pharmaceutical composition. Exemplary excipients include, without limitation, carbohydrates, starches (e.g., corn starch), inorganic salts, antimicrobial agents, antioxidants, binders/fillers, surfactants, lubricants (e.g., calcium or magnesium stearate), glidants (e.g., talc), disintegrants, diluents, buffers, acids, bases, film coats, combinations thereof, and the like.

A composition of the invention may include one or more carbohydrates such as a sugar, a derivatized sugar such as an alditol, aldonic acid, an esterified sugar, and/or a sugar polymer. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, sorbitol (glucitol), pyranosyl sorbitol, myoinositol, and the like.

Also suitable for use in the compositions of the invention are potato and corn-based starches such as sodium starch glycolate and directly compressible modified starch.

Further representative excipients include inorganic salts or buffers such as citric acid, sodium chloride, potassium chloride, sodium sulfate, potassium nitrate, sodium phosphate monobasic, sodium phosphate dibasic, and combinations thereof.

The composition may also include an antimicrobial agent, e.g., for preventing or deterring microbial growth. Non-limiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

A composition of the invention may also contain one or more antioxidants. Antioxidants are used to prevent oxidation, thereby preventing the deterioration of the drug(s) or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfate, sodium formaldehyde sulfoxylate, sodium metabisulfite, and combinations thereof.

Additional excipients include surfactants such as polysorbates, e.g., “Tween 20” and “Tween 80,” and pluronics such as F68 and F88 (both of which are available from BASF, Mount Olive, N.J.), sorbitan esters, lipids (e.g., phospholipids such as lecithin and other phosphatidylcholines, and phosphatidylethanolamines), fatty acids and fatty esters, steroids such as cholesterol, and chelating agents, such as EDTA, zinc and other such suitable cations.

Further, a composition of the invention may optionally include one or more acids or bases. Non-limiting examples of acids that can be used include acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of any individual excipient in the composition will vary depending on the role of the excipient, the dosage requirements of the active agent components, and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects.

Generally, however, the excipient will be present in the composition in an amount of about 1% to about 99% by weight, preferably from about 5% to about 98% by weight, more preferably from about 15% to about 95% by weight of the excipient, with concentrations less than 30% by weight most preferred.

These foregoing pharmaceutical excipients along with other excipients are described in “Remington: The Science & Practice of Pharmacy”, 19^{th} ed., Williams & Williams, 1995; “Physician's Desk Reference”, 52^{nd} ed., Medical Economics, Montvale, N.J., 1998; and Kibbe A H, Handbook of Pharmaceutical Excipients, 3^{rd} ed., American Pharmaceutical Association, Washington D.C., 2000.

Delivery Forms: The compositions encompass all types of formulations and in particular those that are suited for oral administration, e.g., tablets, lozenges, capsules, syrups, oral suspensions, emulsions, granules, and pellets. Alternative formulations include aerosols, transdermal patches, gels, creams, ointments, suppositories, powders or lyophilates that can be reconstituted, as well as liquids, such as for use in an oral or parenteral product. Examples of suitable diluents for reconstituting solid compositions, e.g., prior to injection, include bacteriostatic water for injection, dextrose 5% in water, phosphate-buffered saline, Ringer's solution, saline, sterile water, deionized water, and combinations thereof. With respect to liquid pharmaceutical compositions, solutions and suspensions are envisioned.

In turning now to oral delivery formulations, tablets can be made by compression or molding, optionally with one or more accessory ingredients or additives. Compressed tablets are prepared, for example, by compressing in a suitable tabletting machine, the active ingredients in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) and/or surface-active or dispersing agent.

Molded tablets are made, for example, by molding in a suitable tabletting machine, a mixture of powdered compounds moistened with an inert liquid diluent. The tablets may optionally be coated or scored, and may be formulated so as to provide slow or controlled release of the active ingredients, using, for example, hydroxypropylmethyl cellulose in varying proportions to provide the desired release profile. Tablets may optionally be provided with a coating, such as a thin film, sugar coating, or an enteric coating to provide release in parts of the gut other than the stomach. Processes, equipment, and toll manufacturers for tablet and capsule making are well-known in the art.

Capsule formulations may utilize either hard or soft capsules, including gelatin capsules or vegetarian capsules such as those made out of hydroxymethylpropylcellulose (HMPC). One preferred type of capsule is a gelatin capsule. Capsules may be filled using a capsule filling machine such as those available from commercial suppliers such as Miranda International or employing capsule manufacturing techniques well-known in the industry, as described in detail in Pharmaceutical Capsules, 2^{nd} ed., F. Podczeck and B. Jones, 2004. Alternatively, capsule formulations may be prepared using a toll manufacturing center such as the Chao Center for Industrial Pharmacy & Contract Manufacturing, located at Purdue Research Park.

Formulations for topical administration in the mouth include lozenges comprising the active ingredients generally in a flavored base such as sucrose and acacia or tragacanth and pastilles comprising the active ingredients in an inert base such as gelatin and glycerin or sucrose and acacia.

A pharmaceutical composition for topical administration may also be formulated as an ointment, cream, suspension, lotion, powder, solution, paste, gel, spray, aerosol or oil. Alternatively, the formulation may be in the form of a patch (e.g., a transdermal patch) or a dressing such as a bandage or adhesive plaster impregnated with active ingredients and optionally one or more excipients or diluents. Topical formulations may additionally include a compound that enhances absorption or penetration of the ingredients through the skin or other affected areas, such as dimethylsulfoxidem bisabolol, oleic acid, isopropyl myristate, and D-limonene, to name a few.

For emulsions, the oily phase is constituted from known ingredients in a known manner. While this phase may comprise merely an emulsifier (otherwise known as an emulgent), it desirably comprises a mixture of at least one emulsifier with a fat and/or an oil. Preferably, a hydrophilic emulsifier is included together with a lipophilic emulsifier that acts as a stabilizer. Together, the emulsifier(s) with or without stabilizer(s) make up the so-called emulsifying wax, and the wax together with the oil and/or fat make up the so-called emulsifying ointment base which forms the oily dispersed phase of cream formulations. Illustrative emulgents and emulsion stabilizers include Tween 60, Span 80, cetostearyl alcohol, myristyl alcohol, glyceryl monostearate and sodium lauryl sulfate.

Formulations for rectal administration are typically in the form of a suppository with a suitable base comprising, for example, cocoa butter or a salicylate.

Formulations suitable for vaginal administration generally take the form of a suppository, tampon, cream, gel, paste, foam or spray.

Formulations suitable for nasal administration, wherein the carrier is a solid, include a coarse powder having a particle size, for example, in the range of about 20 to about 500 microns. Such a formulation is typically administered by rapid inhalation through the nasal passage, e.g., from a container of the powder held in proximity to the nose. Alternatively, a formulation for nasal delivery may be in the form of a liquid, e.g., a nasal spray or nasal drops.

Aerosolizable formulations for inhalation may be in dry powder form (e.g., suitable for administration by a dry powder inhaler), or, alternatively, may be in liquid form, e.g., for use in a nebulizer. Nebulizers for delivering an aerosolized solution include the AERx™ (Aradigm), the Ultravent® (Mallinkrodt), and the Acorn II® (Marquest Medical Products). A composition of the invention may also be delivered using a pressurized, metered dose inhaler (MDI), e.g., the Ventolin® metered dose inhaler, containing a solution or suspension of a combination of drugs as described herein in a pharmaceutically inert liquid propellant, e.g., a chlorofluorocarbon or fluorocarbon.

Formulations suitable for parenteral administration include aqueous and non-aqueous isotonic sterile solutions suitable for injection, as well as aqueous and non-aqueous sterile suspensions.

Parenteral formulations of the invention are optionally contained in unit-dose or multi-dose sealed containers, for example, ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the types previously described.

A formulation of the invention may also be a sustained release formulation, such that the fatty acid oxidation inhibitor and/or carbohydrate oxidation activator is released or absorbed slowly over time, when compared to a non-sustained release (immediate release) formulation. Sustained release formulations may employ pro-drug forms of the active agent, delayed-release drug delivery systems such as liposomes or polymer matrices, hydrogels, or covalent attachment of a polymer such as polyethylene glycol to the active agent.

In addition to the ingredients particularly mentioned above, the formulations of the invention may optionally include other agents conventional in the pharmaceutical arts and particular type of formulation being employed, for example, for oral administration forms, the composition for oral administration may also include additional agents as sweeteners, thickeners or flavouring agents.

### Kits

Also provided herein is a kit or package containing a fatty acid oxidation inhibitor and/or a carbohydrate oxidation activator, in packaged form, accompanied by instructions for use. The fatty acid oxidation inhibitor and/or carbohydrate oxidation activator may be packaged in any manner suitable for administration, so long as the packaging, when considered along with the instructions for administration, clearly indicates the manner in which the fatty acid oxidation inhibitor and/or carbohydrate oxidation activator is to be administered.

For example, the kit comprises a fatty acid oxidation inhibitor and/or a carbohydrate oxidation activator in unit dosage form, along with instructions for use. For example, such instructions may indicate that administration of the fatty acid oxidation inhibitor and/or carbohydrate oxidation activator is useful in the treatment of conditions such as one or more of the following: FMS, CGS, MPS, MCS and GWS. The fatty acid oxidation inhibitor and/or carbohydrate oxidation activator may be packaged in any manner suitable for administration, so long as the packaging, when considered along with the instructions for administration, clearly indicates the manner in which the drug component is to be administered. For example, when a fatty acid oxidation inhibitor and/or a carbohydrate oxidation activator is in oral dosage form, e.g., is in the form of a coated tablet, then the kit may comprise a sealed container of coated tablets, blister strips containing the tablets, or the like. Other preferred dosage forms include capsules, syrups and suspensions. In some embodiments, for oral dosage forms, it is recommended that the fatty acid oxidation inhibitor and/or carbohydrate oxidation activator be administered after meals.

Various embodiments according to the above may be readily envisioned, and would of course depend upon the particular dosage form, recommended dosage, intended patient population, and the like. The packaging may be in any form commonly employed for the packaging of pharmaceuticals, such as medication punch cards or blisters, and may utilize any of a number of features such as different colors, wrapping, tamper-resistant packaging, blister packs or strips, desiccants, and the like.

### Method of Administration

As set forth above, methods of delivery include but are not limited to, oral, intra-arterial, intramuscular, intravenous, intranasal, and inhalation routes. A preferred delivery route is oral. Suitable modes of delivery will be apparent based upon the particular combination of drugs employed and their known administration forms.

More particularly, a fatty acid oxidation inhibitor and/or a carbohydrate oxidation activator for use in treating a condition such as FMS, CFS, MPS, MCS, and GWS, may be administered by any suitable route, including without limitation, oral, rectal, nasal, topical (including transdermal, aerosol, buccal and sublingual), vaginal, penile, parenteral (including subcutaneous, intramuscular, intravenous and intradermal) and pulmonary. The preferred route will, of course, vary with the condition and age of the recipient, and the particular condition being treated.

A fatty acid oxidation inhibitor and/or a carbohydrate oxidation activator may be administered in unit dosage form multiple times daily, but most preferably is administered once, twice or three times daily. In terms of patient compliance and ease of administration, such an approach is preferred over more frequent dosing, since patients are often adverse to taking multiple pills or capsules, often multiple times daily, over the duration of treatment. In instances in which a fatty acid oxidation inhibitor and/or a carbohydrate oxidation activator is co-administered along with another active agent as separate dosage forms, each of the different active agents may be administered simultaneously, sequentially in any order, or separately.

### Dosages and Measurable Result

Therapeutic amounts can be empirically determined and will vary with the particular condition being treated, the subject, the particular formulation components, dosage form, and the like. The actual dose to be administered will vary depending upon the age, weight, and general condition of the subject, the particular fatty acid oxidation inhibitor and/or carbohydrate oxidation activator to be used, as well as the severity of the condition being treated, along with the judgment of the health care professional.

Therapeutically effective amounts can be determined by those skilled in the art, and will be adjusted to the requirements of each particular case. Generally, a therapeutically effective amount of a fatty acid oxidation inhibitor and/or a carbohydrate oxidation activator will range from about 1 milligram to about 500 milligrams daily. Preferably, a therapeutically effective amount of a fatty acid oxidation inhibitor and/or a carbohydrate oxidation activator for the treatment of any one or more of FMS, CFS, MPS, MCS, and GWS, is in a range from about 10 milligrams to about 200 milligrams daily, or even more preferably from about 20 milligrams to about 100 milligrams daily. Unit dosage forms of a fatty acid oxidation inhibitor and/or a carbohydrate oxidation activator may be formulations comprising one quarter, one third, one half or the entire daily therapeutically effective amount, although additional dosage forms are envisioned.

Representative dosages of a fatty acid oxidation inhibitor and/or a carbohydrate oxidation activator are typically selected from the group consisting of: from about 5 to about 100 mg/twice daily, from about 10 to about 50 mg/twice daily, from about 20 to about 40 mg/twice daily, from about 10 to about 50 mg/three times daily, from about 20 to about 40 mg/three times daily, about 35 mg/three times daily, and about 35 mg/twice daily, among others.

Practically speaking, a unit dose of any given fatty acid oxidation inhibitor and/or carbohydrate oxidation activator composition of the invention can be administered in a variety of dosing schedules, depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, every other day, three times weekly, twice weekly, once weekly, twice monthly, once monthly, and so forth.

The duration of treatment will depend of course on the particular condition, its severity, the age and condition of the patient, and the like, and will be readily determined by one of skill in the art. Illustrative courses of therapy include 1 week, 2 weeks, 3 weeks, 4 weeks, 5 weeks, 6 weeks, 7 weeks, 8 weeks, 9 weeks, 10 weeks, 11 weeks, 12 weeks, 3.5 months, 4 months, 4.5 months, 5 months, 6 months, 9 months, a year, or longer as needed. Treatment is typically continued until at least a 50% improvement is effected in one or more major symptoms associated with the particular condition being treated.

For example, in treating a subject suffering from FMS, treatment is generally continued until at least a 50% improvement is effected, e.g., in widespread pain of at least three anatomical sites of the subject's body, or in trigger point tenderness. Additionally, improvement may also be noted in one or more minor symptoms experienced by a subject suffering from FMS, e.g., fatigue, irritable bowel syndrome, sleep disorder, chronic headaches, jaw pain, cognitive or memory impairment, post-exertional malaise and muscle pain, morning stiffness, menstrual cramping, numbness and tingling sensations, dizziness, or chemical sensitivities. Preferably, treatment is continued until the subject experiences an improvement of at least about 60%, and more preferably at least about 70%, and even more preferably at least about 80%, and even more preferably 90% or greater in at least one major symptom associated with the condition, e.g., FMS, and additionally, experiences a degree, e.g., 50% or greater, of improvement in one or more associated minor symptoms. Most preferably, treatment is generally continued until substantial resolution of all symptoms is effected or until the patient reports (or the physician notes) either no further improvement, or only minor or insignificant improvement in the subject's remaining symptoms with continued therapy as described herein.

The foregoing applies to each of the conditions described herein as treatable by administration of a fatty acid oxidation inhibitor and/or a carbohydrate oxidation activator.

For example, in the instance of a subject suffering from CFS, treatment is effected until the subject reports a lessening of at least about 50% in the degree of persistent fatigue experienced. Preferably, treatment is continued until the subject experiences an improvement of at least about 60%, and more preferably at least about 70%, and even more preferably at least about 80%, and even more preferably 90% or greater in the degree of persistent fatigue experienced. Preferably, therapeutic treatment with a fatty acid oxidation inhibitor and/or a carbohydrate oxidation activator is also effective to provide improvement in one or more minor CFS-associated symptoms experienced by the subject, e.g., impaired memory or concentration, tender cervical or axillary lymph nodes in the neck region, sore throat, muscle pain, multi-joint pain, headaches, unrefreshing sleep, and post-exertional malaise. Again, such improvement will typically be of a degree of at least about 50% improvement or greater. Ideally, treatment with a fatty acid oxidation inhibitor and/or a carbohydrate oxidation activator is effective to result in improvement of at least about 60%, or at least about 70%, or even more preferably at least about 80%, or most preferably 90% or greater of said one or more minor symptoms.

In the instance of a subject suffering from MPS, treatment is effected until the subject reports a lessening of the degree of pain in one or more trigger points of at least about 50%. Preferably, treatment is continued until the subject experiences an improvement of at least about 60%, and more preferably at least about 70%, and even more preferably at least about 80%, and even more preferably 90% or greater in the degree of muscle pain experienced in one or more trigger points. Preferably, therapeutic treatment with a fatty acid oxidation inhibitor and/or a carbohydrate oxidation activator is also effective to provide improvement in one or more minor MPS-associated symptoms experienced by the subject, e.g., numbness, dizziness, headache, concentration and memory impairment, sleep disorders, fluid retention, balance problems (instability), and stiffness. Again, such improvement will typically be of a degree of at least about 50% improvement or greater. Ideally, treatment with a fatty acid oxidation inhibitor and/or a carbohydrate oxidation activator is effective to result in improvement of at least about 60%, or at least about 70%, or even more preferably at least about 80%, or most preferably 90% or greater of said one or more minor symptoms associated with MPS.

Treatment of a subject suffering from GWS or from MCS with a fatty acid oxidation inhibitor and/or a carbohydrate oxidation activator is over a duration of time effective to result in a similar degree of improvement of associated major and preferably minor symptoms associated therewith as described above.

In one embodiment, in relation to any of the above-mentioned disorders, the treatment with a fatty acid oxidation inhibitor and/or a carbohydrate oxidation activator results in the disappearance of at least 50% of the total number of symptoms, trigger points and/or painful tender points. In another embodiment, the treatment with a fatty acid oxidation inhibitor and/or a carbohydrate oxidation activator results in the disappearance of at least 60% of the total number of symptoms, trigger points and/or painful tender points, preferably at least 70%, preferably at least 80%, preferably at least 90% of the total number of symptoms, trigger points and/or painful tender points.

It is to be understood that while the invention has been described in conjunction with preferred specific embodiments, the foregoing description as well as the examples that follow are intended to illustrate and not limit the scope of the invention. Other aspects, advantages and modifications within the scope of the invention will be apparent to those skilled in the art to which the invention pertains.

### Abbreviations

The following is a list of abbreviations that are used herein.
FMS fibromyalgia syndrome
CFS chronic fatigue syndrome
MCS multiple chemical sensitivities
MPS myofascial pain syndrome
GWS Gulf War syndrome
CBC complete blood count
ESR erythrocyte sedimentation rate
CRP C-reactive protein
ANA antinuclear antibody
OTC over the counter
NSAID non-steroidal anti-inflammatory drug
MR modified release
TP trigger point
DSM-IV Diagnostic and Statistical Manual of Mental Disorders
SCID structured clinical interview for DSM-IV

All articles, books, patents and other publications referenced herein are hereby incorporated by reference in their entireties.

### EXAMPLES

### Example 1

### Treatment of Human Subject Diagnosed with Fibromyalgia with Trimetazidine

A 40 year old male presented with long-standing complaints (15 years) of widespread pain—headaches, upper and lower back pain, neck pain, elbow and knee pain bilaterally, calf pain bilaterally and jaw pain. General non-specific symptoms were fatigue, symptoms of irritable bowel syndrome (constipation-type), insomnia, cognitive and memory impairment, morning stiffness, dizziness, irritability and depression.

Physical Examination: A physical examination revealed tender points located at:
(i) the sub-occipital muscle insertions bilaterally, at the occiput
(ii) the lateral epicondyle at the right elbow
(iii) the medial fat pads of the knees, just proximal to the joint lines, bilaterally
(iv) the trapezius muscles, at the midpoint of the upper border of the muscles, bilaterally
(v) the supraspinatus muscles, at the origins, above the spine of the scapula near the medial border, bilaterally.

Laboratory Tests: The following laboratory investigations were conducted: CBC, ESR, CRP, Rheumatoid Factor, Thyroid Function Tests, ANA. These tests yielded no positive results. X-Rays were also taken and resulted in no positive findings.

Patient History of Prior Treatment: A patient history was taken. Prior treatments targeting pain relief were as follows. OTC analgesics such as paracetamol (also known as acetaminophen), NSAIDs and low dose codeine provided no pain relief whatsoever. Prescription analgesics such as high dose codeine, tramadol and meperidine provided, at best, mild to moderate and very temporary relief and were frequently accompanied by unpleasant side effects. Trigger point injections with lidocaine or lidocaine mixed with triamcinolone (a corticosteroid) provided the best localized pain relief. However, these injections often had to be repeated at 2 to 4 week intervals, and were usually extremely painful procedures.

Prior treatments targeting other symptoms included the following. The subject had taken tegaserod for irritable bowel syndrome. Additional prior therapies included sertraline for depression, and zolpidem for insomnia. The subject has also taken proclorperazine for dizziness.

All of the above therapies were either minimally effective or completely ineffective.

Diagnosis: The subject was diagnosed with fibromyalgia syndrome.

Relevant Additional Medical History: The subject also has cardiac tachyarrhythmia (likely supraventricular tachycardia) with paroxysmal episodes of chest pains and syncope—currently in the process of being investigated.

Treatment Regime: The subject was administered trimetazidine (modified release formulation, Vastarel® MR), 35 mg orally, twice daily, originally for treatment of his cardiac condition.

Results: Within 72 hours of starting treatment, there was a surprising and completely unexpected and vast improvement in all symptoms and signs of fibromyalgia. The benefits were first noticed within 24 hours of commencing therapy and further improved gradually thereafter. Positive results were as follows. The subject reported a “90% reduction” in the widespread pain, with minimal residual pain reported at the left medial knee. The subject additionally reported very significantly increased energy levels, very significantly improved sleep patterns (described as “uninterrupted and very restful”), markedly improved regularity of bowel habits, markedly increased clarity of thoughts and improvement in short and long term memory, and very improved mood and markedly reduced level of irritability.

Follow-up. After six (6) months of continuous use of trimetazidine (modified release) 35 mg, orally, twice daily, the patient has reported that he continues to experience the same marked improvement in the quality of his life, with infrequent episodes of pain at his trigger points, which usually occurs in reaction to stressful episodes or concurrent bacterial or viral infections and usually reduces once again to negligible levels after these circumstances or conditions resolve.

### Example 2

### Treatment of Human Subject Diagnosed with Fibromyalgia with Trimetazidine

A 35 year old female presented with long-standing complaints (10 years) of widespread pain—headaches, upper and lower back pain, neck pain, hip pain bilaterally, knee pain bilaterally and jaw pain. General non-specific symptoms were fatigue, symptoms of irritable bowel syndrome (constipation-type), insomnia, cognitive and memory impairment, morning stiffness, and depression.

Physical Examination: A physical examination revealed tender points located at:
(i) the sub-occipital muscle insertions bilaterally, at the occiput
(ii) the medial fat pads of the knees, just proximal to the joint lines, bilaterally
(iii) the trapezius muscles, at the midpoint of the upper border of the muscles, bilaterally
(iv) greater trochanter: bilateral, posterior to the trochanteric prominence
(v) the supraspinatus muscles, at the origins, above the spine of the scapula near the medial border, bilaterally.

Laboratory Tests: The following laboratory investigations were conducted: CBC, ESR, CRP, Rheumatoid Factor, Thyroid Function Tests, ANA. These tests yielded no positive results. X-Rays were also taken and resulted in no positive findings.

Patient History of Prior Treatment: A patient history was taken. Prior treatments targeting pain relief were as follows. OTC analgesics such as paracetamol (also known as acetaminophen), NSAIDs and low dose codeine provided no pain relief whatsoever. Prescription analgesics such as high dose codeine, tramadol and meperidine provided, at best, mild to moderate and very temporary relief and were frequently accompanied by unpleasant side effects. Trigger point injections with lidocaine or lidocaine mixed with triamcinolone (a corticosteroid) provided the best localized pain relief. However, these injections often had to be repeated at 2 to 4 week intervals, and were usually extremely painful procedures.

Prior treatments targeting other symptoms included the following. The subject had taken tegaserod for irritable bowel syndrome. Additional prior therapies included sertraline for depression, and zolpidem for insomnia.

All of the above therapies were either minimally effective or completely ineffective.

Diagnosis: The subject was diagnosed with fibromyalgia syndrome.

Treatment Regime: The subject was administered trimetazidine (modified release formulation, Vastarel® MR), 35 mg orally, twice daily.

Results: Within 24 hours of starting treatment, there was vast improvement in all symptoms and signs of fibromyalgia. Positive results were as follows. The subject reported a “95% reduction” in the widespread pain, with minimal residual pain reported at the left hip. The subject additionally reported very significantly increased energy levels, very significantly improved sleep patterns (described as “uninterrupted and very restful”), markedly improved regularity of bowel habits, markedly increased clarity of thoughts and improvement in short and long term memory, and very improved mood.

Follow-up. After four (4) months of continuous use of trimetazidine (modified release) 35 mg, orally, twice daily, the patient has reported that she continues to experience the same marked improvement in the quality of her life. She has had no relapse of symptoms to date.

### Example 3

### Treatment of Human Subject Diagnosed with Fibromyalgia with Trimetazidine

A 32 year old female presented with a 5 year history of complaints of widespread pain—headaches, upper and lower back pain, neck pain, hip pain bilaterally and knee pain bilaterally. General non-specific symptoms were fatigue, symptoms of irritable bowel syndrome (constipation-type), insomnia, morning stiffness and emotional liability.

Physical Examination: A physical examination revealed tender points located at:
(i) the sub-occipital muscle insertions bilaterally, at the occiput
(ii) the medial fat pads of the knees, just proximal to the joint lines, bilaterally
(iii) the trapezius muscles, at the midpoint of the upper border of the muscles, bilaterally
(iv) the greater trochanter, posterior to the trochanteric prominence, bilaterally
(v) the supraspinatus muscles, at the origins, above the spine of the scapula near the medial border, bilaterally.

Laboratory Tests: The following laboratory investigations were conducted: CBC, ESR, CRP, Rheumatoid Factor, Thyroid Function Tests, ANA. These tests yielded no positive results. X-Rays were also taken and resulted in no positive findings.

Patient History of Prior Treatment: A patient history was taken. Prior treatments targeting pain relief were as follows. OTC analgesics such as paracetamol (also known as acetaminophen), NSAIDs and low dose codeine provided negligible pain relief. Prescription analgesics such as high dose codeine, tramadol and meperidine provided, at best, mild to moderate and very temporary relief and were frequently accompanied by unpleasant side effects. Trigger point injections were refused by the patient.

Prior treatments targeting other symptoms included the following. The subject had taken tegaserod for irritable bowel syndrome. Additional prior therapies included zolpidem, midazolam, zopiclone, and chlorpheniramine for insomnia.

All of the above therapies were either minimally effective or completely ineffective.

Diagnosis: The subject was diagnosed with fibromyalgia syndrome.

Treatment Regime: The subject was administered trimetazidine (modified release formulation, Vastarel® MR), 35 mg orally, twice daily.

Results: Within 24 hours of starting treatment, there was vast improvement in all symptoms and signs of fibromyalgia. Positive results were as follows. The subject reported a “95% reduction” in the widespread pain, with a residual mild headache. The subject additionally reported very significantly increased energy levels, very significantly improved sleep patterns (described as “uninterrupted and very restful”), markedly improved regularity of bowel habits, and very improved mood.

Follow-up. After four (4) months of continuous use of trimetazidine (modified release) 35 mg, orally, twice daily, the patient has reported that she continues to experience the same marked improvement in the quality of her life. She has experienced no relapse of symptoms to date.

### Example 4

### Treatment of Eight (8) Human Subjects Diagnosed with Fibromyalgia with Trimetazidine

The following is a synopsis of the clinical response of each of eight (8) patients diagnosed with FMS, each of whom received treatment with trimetazidine MR (Vastarel® MR), 35 mg orally, twice daily, for a period of at least four (4) weeks.

TABLE 1
Summary of Treatment of 8 FMS Patients With Trimetazidine

| | | | | | | RESPONSE TO | |
|---|---|---|---|---|---|---|---|
| | | AGE OF | | | RESPONSE TO | TRIMETAZIDINE | |
| | | ONSET OF | SUMMARY OF | | TRIMETAZIDINE | (Medium Term | |
| | | FMS | FMS SIGNS | PREVIOUS | (Short Term i.e. | i.e. over four | EXAMINATION |
| AGE | SEX | SYMPTOMS | & SYMPTOMS | TREATMENTS | within 72 hours) | (4) weeks) | FINDINGS |
| 40 yrs | Female | 16 yrs old | Widespread Pain | Muscle Relaxant | 90% reduction in | After four (4) | Sustained |
| | | | Multiple Severe | Antidepressants | widespread pain | months of daily | resolution of all |
| | | | Trigger Point Pain & | OTC Analgesics | and trigger point | use, patient | trigger point |
| | | | Tenderness - | Opiate Analgesics | pain. | reported a 95% | tenderness. |
| | | | especially at lateral | Trigger Point | Significantly | sustained | |
| | | | epicondyle & bilateral | Injections | improved mood | improvement of | |
| | | | suboccipital | Gastric Acid | and concentration. | symptoms. | |
| | | | Depression | Suppressants | Infrequent | Residual symptoms - | |
| | | | Dizziness | Vestibular | dizziness/dizzy | include mild acid | |
| | | | Impaired | Sedatives | spells | reflux and | |
| | | | Concentration | | 90% reduction in | occasional | |
| | | | Irritable Bowel | | IBS and Acid | infrequent | |
| | | | Syndrome | | Reflux Symptoms | headaches | |
| | | | Acid Reflux | | | | |

| | | | | | | RESPONSE TO | |
|---|---|---|---|---|---|---|---|
| | | AGE OF | | | RESPONSE TO | TRIMETAZIDINE | |
| | | ONSET OF | SUMMARY OF | | TRIMETAZIDINE | (Medium/Long Term | |
| | | FMS | FMS SIGNS | PREVIOUS | (Short Term i.e. | i.e. over four | EXAMINATION |
| AGE | SEX | SYMPTOMS | & SYMPTOMS | TREATMENTS | within 72 hours) | (4) weeks) | FINDINGS |
| 32 yrs | Female | 24 yrs old | Widespread Pain | Muscle Relaxant | 90% reduction in | After six (6) weeks | Sustained |
| | | | Trigger Point Pain & | Antidepressants | widespread pain | of daily use, patient | resolution of all |
| | | | Tenderness in mid & | OTC Analgesics | and trigger point | reported a dramatic | trigger point |
| | | | lower back & medial | Opiate Analgesics | pain. | (95%) sustained | tenderness. |
| | | | aspect of ® knee | Trigger Point | Significantly | improvement of | |
| | | | Severe headaches | Injections | improved mood | symptoms. | |
| | | | Fatigue | Vestibular | and concentration. | Residual symptoms | |
| | | | Depression | Sedatives | Infrequent | include occasional | |
| | | | Sleep Disturbance | Yoga | dizziness/dizzy | insomnia. | |
| | | | Dizziness | | spells | | |
| | | | Impaired | | Consistent restful | | |
| | | | Concentration | | sleep | | |
| | | | Disorientation | | | | |
| 36yrs | Female | 32 yrs old | Widespread Pain | Antidepressants | 90% reduction in | After seven (7) | Not Applicable |
| | | | No identifiable | OTC Analgesics | widespread pain | weeks of daily use, | |
| | | | Trigger Point Pain or | Opiate Analgesics | Significantly | patient reported a | |
| | | | Tenderness | Vestibular | improved mood | dramatic 95% | |
| | | | Joint Pain | Sedatives | and concentration. | sustained | |
| | | | Severe headaches | Physiotherapy | Infrequent | improvement of | |
| | | | Fatigue | Acupuncture | dizziness/dizzy | symptoms with no | |
| | | | Depression | Anti-Spasmodics | spells | specific | |
| | | | Sleep Disturbance | | Consistent restful | complaints. | |
| | | | Dizziness | | sleep | | |
| | | | Impaired Memory | | 70% reduction in | | |
| | | | Word Finding | | severity of Pre- | | |
| | | | Difficulty | | Menstrual Cramps | | |
| | | | Severe Pre- | | | | |
| | | | Menstrual Cramps | | | | |
| 37 yrs | Male | 19 yrs old | Widespread Pain | Antidepressants | 90% reduction in | After four (4) | Not Applicable |
| | | | Multiple Trigger | OTC Analgesics | widespread pain | weeks of daily use, | |
| | | | Points especially at | Muscle Relaxants | Significantly | patient reported a | |
| | | | the right wrist and | | improved mood | dramatic 90% | |
| | | | both knees | | and concentration. | sustained | |
| | | | Joint Pain | | Infrequent | improvement of | |
| | | | Fatigue | | dizziness/dizzy | symptoms with no | |
| | | | Depression | | spells | specific | |
| | | | Sleep Disturbance | | Consistent restful | complaints. | |
| | | | Dizziness | | sleep | | |
| | | | Impaired Memory | | Energetic feeling | | |
| | | | Word Finding | | | | |
| | | | Difficulty | | | | |
| 48 yrs | Female | 37 yrs old | Widespread Pain | Antidepressants | 90% reduction in | After nine (9) | Sustained |
| | | | Multiple Trigger | OTC Analgesics | widespread pain | weeks of daily use, | resolution of |
| | | | Points especially in | Muscle Relaxants | Significantly | patient reported a | Trigger point |
| | | | the upper and lower | Anti-Emetics | improved mood | 90% sustained | tenderness and |
| | | | back and hips | Opiate Analgesics | and concentration. | improvement of | markedly improved |
| | | | Spinal & Joint Pain | Sedatives and | Infrequent dizzy | symptoms with | affect |
| | | | Fatigue | Hypnotics | spells | residual infrequent | |
| | | | Depression/Nervousness | Analgesic | Consistent restful | episodes of joint | |
| | | | Sleep Disturbance | Adjuncts/ | sleep | pains only | |
| | | | Blurred Vision | Anticonvulsants | No Nervousness | | |
| | | | Nausea & Vomiting | | Absence of Nausea | | |
| | | | Impaired Memory | | and Vomiting | | |
| | | | Generalized stiffness | | Improved Memory | | |
| | | | and tightness of the | | 90% reduction of | | |
| | | | muscles, worse in | | feeling of | | |
| | | | the a.m. | | generalized | | |
| | | | | | tightness and | | |
| | | | | | stiffness of muscles | | |
| | | | | | Energetic feeling | | |
| 37 yrs | Female | 31 yrs old | Widespread Pain | Antidepressants | 90% reduction in | After five (5) | Sustained |
| | | | Multiple Trigger | OTC Analgesics | widespread pain | weeks of daily use, | resolution of |
| | | | Points esp. in the | Muscle Relaxants | Significantly | patient reported a | Trigger point |
| | | | upper chest, the | Anti-Emetics | improved mood | dramatic 95% | tenderness and |
| | | | lower back, hips and | Opiate Analgesics | and memory | sustained | markedly improved |
| | | | knees & gen. joint | Sedatives and | Absence of | improvement of all | Affect |
| | | | pains | Hypnotics | dizziness/dizzy | symptoms and | |
| | | | Restless Leg | Analgesic | spells | specifically an | |
| | | | Syndrome | Adjuncts/ | Consistent restful | increased sense of | |
| | | | Headaches & Nausea | Anticonvulsants | sleep | well-being | |
| | | | Burning sensation of | | Absence of Nausea | | |
| | | | the upper limbs and | | 90% reduction in | | |
| | | | the soles of the feet | | feeling of | | |
| | | | bilaterally and | | generalized | | |
| | | | increased sensitivity | | tightness and | | |
| | | | to cold | | stiffness of muscles | | |
| | | | Depression/Impaired | | Energetic feeling | | |
| | | | Memory & Fatigue | | Increased alertness | | |
| | | | Sleep Disturbance | | and clarity of | | |
| | | | Generalized stiffness | | thinking | | |
| | | | and tightness of the | | | | |
| | | | muscles, worse in | | | | |
| | | | the a.m. | | | | |
| 26 yrs | Female | 20 yrs old | Widespread Pain | Antidepressants | 90% reduction in | After four (4) | Sustained |
| | | | Multiple Trigger | OTC Analgesics | widespread pain | weeks of daily use, | resolution of |
| | | | Points esp. in the | Muscle Relaxants | Significantly | patient reported a | trigger point |
| | | | right elbow, behind | Opiate Analgesics | improved mood | dramatic 95% | tenderness and |
| | | | the neck and upper | Sedatives and | and memory | sustained | markedly improved |
| | | | back bilaterally and | Hypnotics | Consistent restful | improvement of all | affect and energy |
| | | | gen. joint pains. | Analgesic | sleep | symptoms and | levels with an |
| | | | Headaches and | Adjuncts/ | 90% reduction in | specifically an | obvious marked |
| | | | Retro-bulbar pain | Anticonvulsants | feeling of | increased sense of | improvement in |
| | | | Difficulty walking | Yoga | generalized | well-being and in | physical |
| | | | due to pain in the | Trigger Point | tightness and | particular a | appearance |
| | | | soles of the feet | injections | stiffness of muscles | sustained increase | |
| | | | Depression/Impaired | Physiotherapy | Energetic feeling | in energy levels | |
| | | | Memory & Fatigue | (muscle | Increased alertness | | |
| | | | Sleep Disturbance | stretching) | and clarity of | | |
| | | | Irritable Bowel | Acid | thinking | | |
| | | | Syndrome & Chest | Suppressants | Markedly reduced | | |
| | | | Pain (Acid Reflux) | Acupuncture | symptoms of | | |
| | | | Generalized stiffness | Homeopathic | Irritable Bowel | | |
| | | | and tightness of the | treatment | Syndrome | | |
| | | | muscles, worse a.m. | | Absence of Chest | | |
| | | | | | Pain | | |
| 54 yrs | Female | 32 yrs old | Widespread Pain in | OTC Analgesics | 90% reduction in | After ten (10) | Markedly |
| | | | particular in the | Muscle Relaxants | widespread pain | weeks of daily use, | improved energy |
| | | | lower back, knees | Sedatives and | 90% reduction in | patient reported a | levels |
| | | | and flanks | Hypnotics | irritability, along | 90 to 95% | |
| | | | bilaterally; often | Anxiolytics | with 90% | improvement of all | |
| | | | worse with exertion | Physiotherapy | improvement in | symptoms. | |
| | | | and at nights & | Laxatives and | mood and memory | A flare up of FMS | |
| | | | gen. joint pain | Anti-Spasmodics | Consistent restful | symptoms occurred | |
| | | | Dizziness | Analgesic | sleep | once with the | |
| | | | Headaches/Retrobulbar | Adjuncts/ | Energetic feeling | occurrence of | |
| | | | pain | Anticonvulsants | Increased alertness | kidney stone pain | |
| | | | Impaired | | and clarity of | (Ureteric Colic) but | |
| | | | Concentration & | | thinking | resolved | |
| | | | Irritable Moods | | 90% reduction in | completely on | |
| | | | Photosensitivity | | symptoms of | passage the kidney | |
| | | | Sleep Disturbance/ | | Irritable Bowel | stone | |
| | | | Fatigue & | | Syndrome | | |
| | | | Nervousness | | | | |
| | | | Irritable Bowel | | | | |
| | | | Syndrome | | | | |
| | | | Chest Pain | | | | |
| | | | Numbness of the | | | | |
| | | | upper limbs | | | | |
| | | | bilaterally | | | | |

1-65. (canceled)

66. A compound, or a pharmaceutically acceptable salt, prodrug or hydrate thereof, for use in the treatment or prophylaxis of
(i) fibromyalgia;
(ii) chronic fatigue syndrome;
(iii) myofascial pain syndrome;
(iv) Gulf War syndrome;
(v) multiple chemical sensitivity;
(vi) widespread pain of at least three anatomical sites of a mammalian subject's body;
(vii) the symptom of unexplained fatigue which is persisting or relapsing;
(viii) the symptom of pain in one or more trigger points; and/or
(ix) one or more symptoms, in a subject that is a veteran of the Gulf War, selected from the group consisting of aching muscles, spasm, fatigue, irritability, thick saliva, weight loss, diarrhoea, skin rash, memory loss, dizziness, peripheral numbness, sleep disturbance, chronic fever, laboured breathing, and headache;
wherein the compound, pharmaceutically acceptable salt, prodrug or hydrate is a fatty acid oxidation inhibitor and/or a carbohydrate oxidation activator.

67. A compound, pharmaceutically acceptable salt, prodrug or hydrate as claimed in claim 66, wherein the fatty acid oxidation inhibitor is a mitochondrial fatty acid oxidation inhibitor.

68. A compound, pharmaceutically acceptable salt, prodrug or hydrate as claimed in claim 67, wherein the mitochondrial fatty acid oxidation inhibitor is a beta-oxidation inhibitor.

69. A compound, pharmaceutically acceptable salt, prodrug or hydrate as claimed in claim 68, wherein the beta-oxidation inhibitor is an inhibitor of acyl-CoA dehydrogenase, enoyl-CoA hydratase, 3-hydroxyacyl-CoA dehydrogenase or 3-ketoacyl-CoA thiolase.

70. A compound, pharmaceutically acceptable salt, prodrug or hydrate as claimed in claim 66, wherein the compound, pharmaceutically acceptable salt, prodrug or hydrate is selected from hypoglycin, 3-chloro-3-butenoylpantetheine, 3-pentenoylpantetheine, iodoacetamide, N-ethylmaleimide, dithioerythritol, EDTA, o-phenanthroline, 2-mercaptoacetate, iodoacetic acid, 1-ethyl-3-(3-dimethylaminopropyl)-carbodiimide, diethyldicarbonate, iodoacetate, salicylic acid, 5,5′-dithiobis(2-nitrobenzoic acid), N-bromosuccinimide, 4-pentenoic acid, 2-bromooctanoic acid, 4-bromocrotonic acid, 4-bromotiglic acid, trimetazidine, ranolazine, 4-bromo-2-octenoic acid, N-methylmaleimide, semicarbazide, tris(hydroxymethyl)aminomethane, benzotript, etomoxir, oxfenicine, 4-hydroxyphenylglyoxylate, 2-tetradecylglycidic acid, trimetazidine derivative S-15176, metoprolol, perhexiline, amiodarone, aminocarnitine, 11-trimethylamino-undecanoyl-DL-carnitine, cardiolipin, carnitine, chenodeoxycholic acid, cholic acid, deoxycarnitine, digitonin, ethyl 2-[6-(4-nitrophenoxy)hexyl]oxirane-2-carboxylate, γ-linolenic acid, hemipalmitoylcarnitinium bromide, L-palmitoylcarnitine, L-sulfocarnitine, octyl glucoside, palmitoylcholine, phosphatidylcholine, 2-(2-naphthalen-2-yloxyethoxy)thiophene-5-glyoxylic acid, thiolcarnitine, 2-(3-methyl-cinnamyl-hydrazono)propionate, 2-(3-phenylpropoxyimino)butyric acid, medium and long-chain acyl carnitines, BM 13.907, 3-(2,2,2-trimethylhydrazine)propionate, 2-oxoglutarate, 3,4-dihydroxybenzoate, 3-(2,2-dimethylcyclopropyl)propionic acid, 3-bromo-2-oxoglutarate, 3-glutathione-2-oxoglutarate, 3-trimethylaminopropyl-1-sulfonate, 2,2′-bipyridine, ascorbate, dioxane, riboflavin-5′-phosphate, iodosobenzoate, pyridine-2,4-dicarboxylate, quinacrine, succinic semialdehyde, dichloroacetate, 3,3,3-trifluoro-2-hydroxy-2-methylpropionamide, 2-chloroisohexanoate, 2-oxobutyrate, dichloroacetophenone, dihydrolipoic acid, 3,3-dichloro-2-benzofuran-1-one, pyruvamide, lipoic acid, rapamycin, thiamine diphosphate, dobutamine, or a pharmaceutically acceptable salt, prodrug or hydrate thereof.

71. A compound, pharmaceutically acceptable salt, prodrug or hydrate as claimed in claim 70, for use in the treatment of
(i) fibromyalgia;
(ii) chronic fatigue syndrome;
(iii) myofascial pain syndrome;
(iv) Gulf War syndrome; and/or
(v) multiple chemical sensitivity;
wherein the treatment results in a diminution of one or more major symptoms associated with said condition.

72. A compound, pharmaceutically acceptable salt, prodrug or hydrate as claimed in claim 71, wherein the treatment further results in a diminution of one or more symptoms selected from: tender point pain and tenderness, depression, dizziness, impaired concentration, irritable bowel syndrome, headache, fatigue, and sleep disturbance.

73. A method for the treatment or prophylaxis of
(i) fibromyalgia;
(ii) chronic fatigue syndrome;
(iii) myofascial pain syndrome;
(iv) Gulf War syndrome;
(v) multiple chemical sensitivity;
(vi) widespread pain of at least three anatomical sites of a mammalian subject's body;
(vii) the symptom of unexplained fatigue which is persisting or relapsing;
(viii) the symptom of pain in one or more trigger points; and/or
(ix) one or more symptoms, in a subject that is a veteran of the Gulf War, selected from the group consisting of aching muscles, spasm, fatigue, irritability, thick saliva, weight loss, diarrhoea, skin rash, memory loss, dizziness, peripheral numbness, sleep disturbance, chronic fever, laboured breathing, and headache;
said method comprising administering to a mammalian subject a therapeutically or prophylactically effective amount of a compound, or a pharmaceutically acceptable salt, prodrug or hydrate thereof, wherein the compound, pharmaceutically acceptable salt, prodrug or hydrate is a fatty acid oxidation inhibitor and/or a carbohydrate oxidation activator.

74. A method as claimed in claim 73, wherein the fatty acid oxidation inhibitor is a mitochondrial fatty acid oxidation inhibitor.

75. A method as claimed in claim 74, wherein the mitochondrial fatty acid oxidation inhibitor is a beta-oxidation inhibitor.

76. A method as claimed in claim 75, wherein the beta-oxidation inhibitor is an inhibitor of acyl-CoA dehydrogenase, enoyl-CoA hydratase, 3-hydroxyacyl-CoA dehydrogenase or 3-ketoacyl-CoA thiolase.

77. A method as claimed in claim 73, wherein the fatty acid oxidation inhibitor is a mitochondrial fatty acid transport inhibitor.

78. A method as claimed in claim 73, wherein the carbohydrate oxidation activator is a glucose oxidation activator.

79. A method as claimed in claim 73, wherein the compound, pharmaceutically acceptable salt, prodrug or hydrate is selected from hypoglycin, 3-chloro-3-butenoylpantetheine, 3-pentenoylpantetheine, iodoacetamide, N-ethylmaleimide, dithioerythritol, EDTA, o-phenanthroline, 2-mercaptoacetate, iodoacetic acid, 1-ethyl-3-(3-dimethylaminopropyl)-carbodiimide, diethyldicarbonate, iodoacetate, salicylic acid, 5,5′-dithiobis(2-nitrobenzoic acid), N-bromosuccinimide, 4-pentenoic acid, 2-bromooctanoic acid, 4-bromocrotonic acid, 4-bromotiglic acid, trimetazidine, ranolazine, 4-bromo-2-octenoic acid, N-methylmaleimide, semicarbazide, tris(hydroxymethyl)aminomethane, benzotript, etomoxir, oxfenicine, 4-hydroxyphenylglyoxylate, 2-tetradecylglycidic acid, trimetazidine derivative S-15176, metoprolol, perhexiline, amiodarone, 11-trimethylamino-undecanoyl-DL-carnitine, cardiolipin, carnitine, chenodeoxycholic acid, cholic acid, deoxycarnitine, digitonin, ethyl 2-[6-(4-nitrophenoxy)hexyl]oxirane-2-carboxylate, γ-linolenic acid, hemipalmitoylcarnitinium bromide, L-palmitoylcarnitine, L-sulfocarnitine, octyl glucoside, palmitoylcholine, phosphatidylcholine, 2-(2-naphthalen-2-yloxyethoxy)thiophene-5-glyoxylic acid, thiolcarnitine, 2-(3-methyl-cinnamyl-hydrazono)propionate, 2-(3-phenylpropoxyimino)butyric acid, medium and long-chain acyl carnitines, BM 13.907, 3-(2,2,2-trimethylhydrazine)propionate, 2-oxoglutarate, 3,4-dihydroxybenzoate, 3-(2,2-dimethylcyclopropyl)propionic acid, 3-bromo-2-oxoglutarate, 3-glutathione-2-oxoglutarate, 3-trimethylaminopropyl-1-sulfonate, 2,2′-bipyridine, ascorbate, dioxane, riboflavin-5′-phosphate, iodosobenzoate, pyridine-2,4-dicarboxylate, quinacrine, succinic semialdehyde, dichloroacetate, 3,3,3-trifluoro-2-hydroxy-2-methylpropionamide, 2-chloroisohexanoate, 2-oxobutyrate, dichloroacetophenone, dihydrolipoic acid, 3,3-dichloro-2-benzofuran-1-one, pyruvamide, lipoic acid, rapamycin, thiamine diphosphate, dobutamine, or a pharmaceutically acceptable salt, prodrug or hydrate thereof.

80. A method as claimed in claim 79, wherein the compound, pharmaceutically acceptable salt, prodrug or hydrate is selected from hypoglycin, salicylic acid, 4-pentenoic acid, 2-bromooctanoic acid, 4-bromocrotonic acid, 4-bromotiglic acid, trimetazidine, ranolazine, etomoxir, oxfenicine, 4-hydroxyphenylglyoxylate, 2-tetradecylglycidic acid, trimetazidine derivative S-15176, metoprolol, perhexiline, amiodarone, aminocarnitine, 2-(3-methyl-cinnamyl-hydrazono)propionate, 2-(3-phenylpropoxyimino)butyric acid, medium and long-chain acyl carnitines, BM 13.907, 3-(2,2,2-trimethylhydrazine)propionate, dichloroacetate, or a pharmaceutically acceptable salt, prodrug or hydrate thereof.

81. A method as claimed in claim 79, wherein the compound, pharmaceutically acceptable salt, prodrug or hydrate is selected from 4-pentenoic acid, 2-bromooctanoic acid, 4-bromocrotonic acid, 4-bromotiglic acid, trimetazidine, ranolazine, or a pharmaceutically acceptable salt, prodrug or hydrate thereof.

82. A method as claimed in claim 79, wherein the compound, pharmaceutically acceptable salt, prodrug or hydrate is selected from trimetazidine, ranolazine, or a pharmaceutically acceptable salt, prodrug or hydrate thereof.

83. A method as claimed in claim 79, for the treatment of
(i) fibromyalgia;
(ii) chronic fatigue syndrome;
(iii) myofascial pain syndrome;
(iv) Gulf War syndrome; and/or
(v) multiple chemical sensitivity;
wherein the compound, pharmaceutically acceptable salt, prodrug or hydrate is administered over a duration of time effective to result in a diminution of one or more major symptoms associated with said condition.

84. A method as claimed in claim 83, wherein said administering is further effective to result in a diminution of one or more symptoms selected from: tender point pain and tenderness, depression, dizziness, impaired concentration, irritable bowel syndrome, headache, fatigue, and sleep disturbance.

85. A kit comprising:
a compound, or a pharmaceutically acceptable salt, prodrug or hydrate thereof, in packaged form, and
instructions for administering said compound, pharmaceutically acceptable salt, prodrug or hydrate for the treatment or prophylaxis of
(i) fibromyalgia;
(ii) chronic fatigue syndrome;
(iii) myofascial pain syndrome;
(iv) Gulf War syndrome;
(v) multiple chemical sensitivity;
(vi) widespread pain of at least three anatomical sites of a mammalian subject's body;
(vii) the symptom of unexplained fatigue which is persisting or relapsing;
(viii) the symptom of pain in one or more trigger points; and/or
(ix) one or more symptoms, in a subject that is a veteran of the Gulf War, selected from the group consisting of aching muscles, spasm, fatigue, irritability, thick saliva, weight loss, diarrhoea, skin rash, memory loss, dizziness, peripheral numbness, sleep disturbance, chronic fever, laboured breathing, and headache;
wherein the compound, pharmaceutically acceptable salt, prodrug or hydrate is a fatty acid oxidation inhibitor and/or a carbohydrate oxidation activator.
